SEQUENCE LISTING (42 sequences)

SEQ ID NO: 1 - DNA, 1352 bp, Homo sapiens
tctatccaggatgtaaatgagcacactgctggcccatgcgcctcggggctgtagagggcagcctcagaggcactgggcattcctggcaccatggatgacgctgctgtcctcaagcgacgaggctacctcctggggataaatttaggagagggctcctatgcaaaagtaaaatctgcttactctgagcgcctgaagttcaatgtggcgatcaagatcatcgaccgcaagaaggcccccgcagacttcttggagaaattccttccccgggaaattgagattctggccatgttaaaccactgctccatcattaagacctacgagatctttgagacatcacatggcaaggtctacatcgtcatggagctcgcggtccagggcgacctcctcgagttaatcaaaacccggggagccctgcatgaggacgaagctcgcaagaagttccaccagctttccttggccatcaagtactgccacgacctggacgtcgtccaccgggacctcaagtgtgacaaccttctccttgacaaggacttcaacatcaagctgtccgacttcagcttctccaagcgctgcctgcgggatgacagtggtcgaatggcattaagcaagaccttctgtgggtcaccagcgtatgcggccccagaggtgctgcagggcattccctaccagcccaaggtgtacgacatctggagcctaggcgtgatcctctacatcatggtctgcggctccatgccctacgacgactccaacatcaagaagatgctgcgtatccagaaggagcaccgcgtcaacttcccacgctccaagcacctgacaggcgagtgcaaggacctcatctaccacatgctgcagcccgacgtcaaccggcggctccacatcgacgagatcctcagccactgctggatgcagcccaaggcacggggatctccctctgtggccatcaacaaggagggggagagttcccggggaactgaacccttgtggacccccgaacctggctctgacaagaagtctgccaccaagctggagcctgagggagaggcacagccccaggcacagcctgagacaaaacccgaggggacagcaatgcaaatgtccaggcagtcggagatcctgggtttccccagcaagccgtcgactatggagacagaggaagggcccccccaacagcctccagagacgcgggcccagtgagcttcttgcggcccagggaatgagatggagctcacggcttaaagcccaagctctgaagaagtcaagggtggagccagagaaggaaggcagtcccagatgagcctctattttcatcagcttcttctctctccccttgaacttggtaacccacatggttc

SEQ ID NO: 2 - DNA, 1254 bp, Homo sapiens
ttgaggacaacgcctgctggcccacatgacggggggatgtagacggcagcggcgccagtcgctcctggcaccatggacgatgccacagtcctaaggaagaagggttacatcgtaggcatcaatcttggcaagggttcctacgcaaaagtcaaatctgcctactctgagcgcctcaagttcaatgtggctgtcaagatcatcgaccgcaagaaaacacctactgactttgtggagagattccttcctcgggagatggacatcctggcaactgtcaaccacggctccatcatcaagacttacgagatctttgagacctctgacggacggatctacatcatcatggagcttggcgtccagggcgacctcctcgagttcatcaagtgccagggagccctgcatgaggacgtggcacgcaagatgttccgacagctctcctccgccgtcaagtactgccacgacctggacatcgtccaccgggacctcaagtgcgagaaccttctcctcgacaaggacttcaacatcaagctgtctgactttggcttctccaagcgctgcctgcgggacagcaatgggcgcatcatcctcagcaagaccttctgcgggtcggcagcatatgcagcccccgaggtgctgcagagcatcccctaccagcccaaggtgtatgacatctggagcctgggcgtgatcctgtacatcatggtctgcggctccatgccctatgacgactccgacatcaggaagatgctgcgtatccagaaggagcaccgtgtggacttcccgcgctccaagaacctgacctgcgagtgcaaggacctcatctaccgcatgctgcagcccgacgtcagccagcggctccacatcgatgagatcctcagccactcgtggctgcagccccccaagcccaagccaacgtcttctgcttccttcaagagggagggggagggcaagtaccgcgctgagtgcaaactggacaccaagacaggcttgaggcccgaccaccggcccgaccacaagcttggagccaaaacccagcaccggctgctggtggtgcccgagaacgagaacaggatggaggacaggctggccgagacctccagggccaaagaccatcacatctccggagctgaggtggggaaagcaagcacctagcatgacaatggccccgttgtgtgtggtgggggtcggggttggggggcatggtgcagtcggccttcacgtaaactaagtaggcaggtaggatctgaagaaggcaca

SEQ ID NO: 3 - DNA, 1058 bp, Homo sapiens
catcctaatacgactcactatagggctcgagcggcgcccgggcaggtgagaatgttctaacgctgggggcggctgcggatgaagtccttggggagaaaaggagcaggccaagggcgatggtggagtagagctgcctctcagaggcagcatgagctgagagggtgataggaaggcggcgctagacagcatggaggactttctgctctccaatgggtaccagctgggcaagaccattggggaagggacctactcaaaagtcaaagaagcattttccaaaaaacaccaaagaaaagtggcaattaaagttatagacaagatgggagggccagaagagtttatccagagattcctccctcgggagctccaaatcgtccgtaccctggaccacaagaacatcatccaggtgtatgagatgctggagtctgccgacgggaaaatctgcctggtgatggagctcgctgagggaggggatgtctttgactgcgtgctgaatggggggccactgcctgaaagccgggccaaggccctcttccgtcagatggttgaggccatccgctactgccatggctgtggtgtggcccaccgggacctcaaatgtgagaacgccttgttgcagggcttcaacctgaagctgactgactttggctttgccaaggtgttgcccaagtcacaccgggagctgagccagaccttctgcggcagtacagcctatgctgcccccgaggtgctgcagggcattccccacgatagcaaaaaaggtgatgtctggagcatgggtgtggtcctgtatgtcatgctctgtgccagcctaccttttgacgacacagacatccccaagatgctgtggcagcagcagaagggggtgtccttccccactcatctgagcatctcggccgattgccaggacctgctcaagaggctcctggaacccgatatgatcctccggccttcaattgaagaagttagttggcatccatggctagcaagcacttgataaaagcaatggcaagtgctctccaataaagtagggggagaaagcaaagcaaacccaaaaaaaa

SEQ ID NO: 4 - protein, 367 aa, Homo sapiens
MDDAAVLKRRGYLLGINLGEGSYAKVKSAYSERLKFNVAIKIIDRKKAPADFLEKFLPREIEILAMLNHCSIIKTYEIFETSHGKVYIVMELAVQGDLLELIKTRGALHEDEARKKFHQLSLAIKYCHDLDVVHRDLKCDNLLLDKDFNIKLSDFSFSKRCLRDDSGRMALSKTFCGSPAYAAPEVLQGIPYQPKVYDIWSLGVILYIMVCGSMPYDDSNIKKMLRIQKEHRVNFPRSKHLTGECKDLIYHMLQPDVNRRLHIDEILSHCWMQPKARGSPSVAINKEGESSRGTEPLWTPEPGSDKKSATKLEPEGEAQPQAQPETKPEGTAMQMSRQSEILGFPSKPSTMETEEGPPQQPPETRAQ

SEQ ID NO: 5 - protein, 358 aa, Homo sapiens
MDDATVLRKKGYIVGINLGKGSYAKVKSAYSERLKFNVAVKIIDRKKTPTDFVERFLPREMDILATVNHGSIIKTYEIFETSDGRIYIIMELGVQGDLLEFIKCQGALHEDVARKMFRQLSSAVKYCHDLDIVHRDLKCENLLLDKDFNIKLSDFGFSKRCLRDSNGRIILSKTFCGSAAYAAPEVLQSIPYQPKVYDIWSLGVILYIMVCGSMPYDDSDIRKMLRIQKEHRVDFPRSKNLTCECKDLIYRMLQPDVSQRLHIDEILSHSWLQPPKPKATSSASFKREGEGKYRAECKLDTKTGLRPDHRPDHKLGAKTQHRLLVVPENENRMEDRLAETSRAKDHHISGAEVGKAST

SEQ ID NO: 6 - protein, 268 aa, Homo sapiens
MEDFLLSNGYQLGKTIGEGTYSKVKEAFSKKHQRKVAIKVIDKMGGPEEFIQRFLPRELQIVRTLDHKNIIQVYEMLESADGKICLVMELAEGGDVFDCVLNGGPLPESRAKALFRQMVEAIRYCHGCGVAHRDLKCENALLQGFNLKLTDFGFAKVLPKSHRELSQTFCGSTAYAAPEVLQGIPHDSKKGDVWSMGVVLYVMLCASLPFDDTDIPKMLWQQQKGVSFPTHLSISADCQDLLKRLLEPDMILRPSIEEVSWHPWLAST

SEQ ID NO: 7 - DNA, 1779 bp, Homo sapiens
atggcgagcgtggtggtgaagacgatctggcagtccaaagagatccatgaggccggggacacccccacgggggtggagagctgctcccagctagtcccagaggctccccggagggtgaccagccgggccaaggggatcccgaagaaaaagaaggccgtgtcgttccacggggtggagccccagatgtcccatcagcccatgcactggtgcctgaacctcaaacggtcctcggcctgcaccaacgtgtcactgctcaacctggccgccatggagcccactgactccacggggacagactccacagtggaagacctcagcggccaactcacactggctgggccccctgcctcccctaccctaccctgggatccggatgacgcagacatcacggaaatcctgagtggggtcaacagtggattggtccgcgccaaagactccatcaccagcttgaaggaaaagaccaaccgggttaaccagcacgtgcagtctctgcagagcgagtgttctgtgctgagcgagaatctggagggaaggcggcaagaggcagaagagttggaggggtactgcattcaactcaaggagaactgctggaaggtgacccggtctgtggaagatgctgaaatcaaaaccaacgtcttgaagcagaattctgccctgctggaggagaagttgcgctacctccagcagcagctgcaggatgagacgccgcgacggcaggaggccgagctgcaggagccggaggagaagcaggagccggaggagaagcaggagccggaggagaagcagaagccggaggctggcctctcctggaacagcctgggccccgccgccacgtcccagggctgccccggcccgccagggagtcccgacaaaccctcgcggccacacggcctggtccccgcaggctggggaatggggcctcgggctggcgagggcccctacgtgagcgagcaggaattgcagaagctgttcaccggcatcgaagagctgaggagagaggtgtcctcactgaccgcccggtggcatcaggaggagggggcggtgcaggaagccctgcggctgctcgggggcctgggcggcagggtcgacggcttcctaggccagtgggagcgggcacagcgcgaacaggcacagacggcgcggggcttgcaggagctgcgaggtcgggcggatgagctgtgcaccatggtggagcggtcagcagtgtctgtggcttcactgaggagcgaactggaggggctgggcccactgaaacccattctggaggagttcgggcggcaatttcagaactctcgaagagggcctgacctctccatgaacctggatcggtcccaccaaggcaactgtgcccgctgtgccagccaggggtcgcagttgtctacggagtccctgcagcagctgctggaccgagcactgacctcactagtggacgaggtgaagcagaggggcctgactcctgcctgtcccagctgtcagaggctacacaagaagattctggagctggagcgccaggccttagccaaacacgtcagggcagaggccctgagctccacccttcggctggcccaagacgaggccctgcgggccaagaacctactgctgacagacaagatgaagccagaggagaagatggccactctggaccatctacacttgaagatgtgctccctccacgatcatctcagcaacctgccacttgaggggtccacgggaacaatggggggaggcagcagtgcaggaacccccccaaaacaggggggctcagcccctgaacaataa

SEQ ID NO: 8 - protein, 592 aa, Homo sapiens
MASVVVKTIWQSKEIHEAGDTPTGVESCSQLVPEAPRRVTSRAKGIPKKKKAVSFHGVEPQMSHQPMHWCLNLKRSSACTNVSLLNLAAMEPTDSTGTDSTVEDLSGQLTLAGPPASPTLPWDPDDADITEILSGVNSGLVRAKDSITSLKEKTNRVNQHVQSLQSECSVLSENLEGRRQEAEELEGYCIQLKENCWKVTRSVEDAEIKTNVLKQNSALLEEKLRYLQQQLQDETPRRQEAELQEPEEKQEPEEKQEPEEKQKPEAGLSWNSLGPAATSQGCPGPPGSPDKPSRPHGLVPAGWGMGPRAGEGPYVSEQELQKLFTGIEELRREVSSLTARWHQEEGAVQEALRLLGGLGGRVDGFLGQWERAQREQAQTARGLQELRGRADELCTMVERSAVSVASLRSELEGLGPLKPILEEFGRQFQNSRRGPDLSMNLDRSHQGNCARCASQGSQLSTESLQQLLDRALTSLVDEVKQRGLTPACPSCQRLHKKILELERQALAKHVRAEALSSTLRLAQDEALRAKNLLLTDKMKPEEKMATLDHLHLKMCSLHDHLSNLPLEGSTGTMGGGSSAGTPPKQGGSAPEQ

SEQ ID NO: 9 - protein, 268 aa, Mus musculus
MEDFLLSNGYQLGKTIGEGTYSKVKEAFSKKHQRKVAIKIIDKMGGPEEFIQRFLPRELQIVRTLDHKNIIQVYEMLESADGKIYLVMELAEGGDVFDCVLNGGPLPESRAKALFRQMVEAIRYCHGCGVAHRDLKCENALLQGFNLKLTDFGFAKVLPKSRRELSQTFCGSTAYAAPEVLQGIPHDSKKGDVWSMGVVLYVMLCASLPFDDTDIPKMLWQQQKGVSFPTHLGISTECQDLLKRLLEPDMILRPSIEEVSWHPWLAST

SEQ ID NO: 10 - DNA, 1020 bp, Mus musculus
gcacgagatgttctagccctggaggcagctgtgaatgaagtccttggggggaaaagaagcaggccgagggcgatggtggagtagagctgcctcgcagaggcagcatgagctgagagggtgacaagaaggaggcgctacacagcatggaggactttctactctccaatgggtatcagctgggcaagaccattggggaagggacctactcaaaagtcaaagaagcattttccaaaaaacatcaacgaaaagtggcaattaaaattatagacaagatgggagggccagaagagtttatccagagattcctgcctcgtgagctccagattgtccgtaccctggaccacaaaaacatcatccaggtgtatgagatgctggagtcagcagatggaaaaatctacctggtgatggagctggctgagggaggggatgtctttgactgtgtgctgaacggagggccacttcccgagagccgggccaaggccctcttccgccagatggttgaggctattcgctattgccatggctgtggcgtggcccaccgggaccttaagtgtgagaacgccttgttgcagggcttcaacctgaagctgaccgactttggctttgccaaggtgctacccaagtcacgcagggagctgagccagaccttctgtggcagcacagcctatgccgcccctgaggtgctacagggcataccccatgatagcaagaaaggtgatgtctggagcatgggtgtggtcctgtatgtaatgctctgtgcaagtctaccttttgacgacacagatatccccaagatgctgtggcagcagcagaagggggtgtccttccccactcatttgggcatctcaaccgaatgccaggacctgctcaagcggctcctggaaccagacatgatactccggccttcaatcgaagaagttagttggcacccatggctagcaagcacttgataaaagcaatggcaagtcctccccaataaagtagggggagaaagcaaactgaaaaaaaaaaaaaaaaaaa

SEQ ID NO: 11 - DNA, 24 bp, Artificial Sequence
  synthetic construct
cgtggatccawaggaccasacrtc

SEQ ID NO: 12 - DNA, 20 bp, Artificial Sequence
  synthetic construct
attcggatccacmgngayyt

SEQ ID NO: 13 - DNA, 24 bp, Artificial Sequence
  synthetic construct
catcacctttcttgctatcatggg

SEQ ID NO: 14 - DNA, 21 bp, Artificial Sequence
  synthetic construct
tgtgagaacgccttgttgcag

SEQ ID NO: 15 - DNA, 23 bp, Artificial Sequence
  synthetic construct
gacatcaccttttttgctatcgt

SEQ ID NO: 16 - DNA, 27 bp, Artificial Sequence
  synthetic construct
ccatcctaatacgactcactatagggc

SEQ ID NO: 17 - DNA, 24 bp, Artificial Sequence
  synthetic construct
gcaggtgagaatgttctaacgctg

SEQ ID NO: 18 - DNA, 24 bp, Artificial Sequence
  synthetic construct
tctccccctactttattggagagc

SEQ ID NO: 19 - DNA, 1467 bp, homo sapiens
acaaagcagggaggctagggtggccccggggcggggtcccggacccccaccctaaggcggagtgacgcccgcagtcacttcacaaggcaaaaattgttacggggcaataaaaggcacaacagcggccaatgtctggcagtgggcacatgggggtgcgggggtgtaggtgccaagcgccatggcttagacccgagattggagtccggccgccccccgacagcagccgcctcctgccccccgtgcgccctaggcgccaccatgtcgggagacaaacttctgagcgaactcggttataagctgggccgcacaattggagagggcagctactccaaggtgaaggtggccacatccaagaagtacaagggtaccgtggccatcaaggtggtggaccggcggcgagcgcccccggacttcgtcaacaagttcctgccgcgagagctgtccatcctgcggggcgtgcgacacccgcacatcgtgcacgtcttcgagttcatcgaggtgtgcaacgggaaactgtacatcgtgatggaagcggccgccaccgacctgctgcaagccgtgcagcgcaacgggcgcatccccggagttcaggcgcgcgacctctttgcgcagatcgccggcgccgtgcgctacctgcacgatcatcacctggtgcaccgcgacctcaagtgcgaaaacgtgctgctgagcccggacgagcgccgcgtcaagctcaccgacttcggcttcggccgccaggcccatggctacccagacctgagcaccacctactgcggctcagccgcctacgcgtcacccgaggtgctcctgggcatcccctacgaccccaagaagtacgatgtgtggagcatgggcgtcgtgctctacgtcatggtcaccgggtgcatgcccttcgacgactcggacatcgccggcctgccccggcgccagaaacgcggcgtgctctatcccgaaggcctcgagctgtccgagcgctgcaaggccctgatcgccgagctgctgcagttcagcccgtccgccaggccctccgcgggccaggtagcgcgcaactgctggctgcgcgccggggactccggctagaagccgggtggttccagccattcctgcagccaagggcactgggccagggcggcgcacgcgcaagaggcgcgcttcgagggaatatgcgaagctgccgcgtgctgctgcacatgcgctttttcccttccgcttccctccctttcttcccacgggggagtccgcagttgcccttgttcggaatccacgttccccgcgatcccgggagctggaggcgcatgcgcatccgcgattccctgcgaccaggccccgagagggcgagaccagaggggacggaagcattgcgcctgcgcggaactctcagcctctgcgcggagggcgtcccttcccaaccagccgtgggtgccaggttcccggttggaacctgcaataaactcgctgttcctcgca

SEQ ID NO: 20 - protein, 273 aa, homo sapiens
MSGDKLLSELGYKLGRTIGEGSYSKVKVATSKKYKGTVAIKVVDRRRAPPDFVNKFLPRELSILRGVRHPHIVHVFEFIEVCNGKLYIVMEAAATDLLQAVQRNGRIPGVQARDLFAQIAGAVRYLHDHHLVHRDLKCENVLLSPDERRVKLTDFGFGRQAHGYPDLSTTYCGSAAYASPEVLLGIPYDPKKYDVWSMGVVLYVMVTGCMPFDDSDIAGLPRRQKRGVLYPEGLELSERCKALIAELLQFSPSARPSAGQVARNCWLRAGDSG

SEQ ID NO: 21 - DNA, 18 bp, Homo sapiens
gcccctaggtggatgagg

SEQ ID NO: 22 - DNA, 19 bp, Homo sapiens
tcacgctctggggggagta

SEQ ID NO: 23 - DNA, 20 bp, Homo sapiens
gggttcctacgcaaaagtca

SEQ ID NO: 24 - DNA, 20 bp, Homo sapiens
gttttcttgcggtcgatgat

SEQ ID NO: 25 - DNA, 20 bp, Homo sapiens
ggggaagggacctactcaaa

SEQ ID NO: 26 - DNA, 20 bp, Homo sapiens
gtccagggtacggacgattt

SEQ ID NO: 27 - DNA, 19 bp, Homo sapiens
tacgcgtcacccgagtgct

SEQ ID NO: 28 - DNA, 18 bp, Homo sapiens
acgcccatgctccacaca

SEQ ID NO: 29 - DNA, 19 bp, Homo sapiens
gctgagcgagaatctggag

SEQ ID NO: 30 - DNA, 20 bp, Homo sapiens
ttcagcatcttccacagacc

SEQ ID NO: 31 - DNA, 20 bp, Homo sapiens
ggattcaaatgaggctccaa

SEQ ID NO: 32 - DNA, 19 bp, Homo sapiens
tggaggtagcgcagcttct

SEQ ID NO: 33 - DNA, 20 bp, Homo sapiens
atcatcagcaatgcctcctg

SEQ ID NO: 34 - DNA, 20 bp, Homo sapiens
atggcatggactgtggtcat

SEQ ID NO: 35 - DNA, 1327 bp, mus musculus
ggtgccaagcaccatgggttaggcccgagattggagtccggccgccccccgacagcagccgcctcctgctccccgcgcgccccgggcgccaccatgtcgggcgacaaactcctgagcgaacttggctataagctgggccgcacgataggtgagggcagctactccaaggtgaaagtggccacctccaagaagtataaagggacggtggccatcaaggtggtggaccgccggcgagcgcctccggacttcgtcaacaagttcctgcctcgagagctgtccatcctgcggggagtacggcacccgcacatcgtacacgttttcgagttcatcgaagtgtgcaatgggaagctgtacatcgtgatggaggcagcagccaccgacctgctgcaggcagtgcagcgcaacgggcgcatcccggggtcgcaggcgcgcgaactcttctcgcagatcgcgggcgccgtgcgctacctgcacgaccaccacctggtgcaccgcgacctcaagtgcgaaaacgtgctgctaagccctgacgagcgccgcgtcaagatcacggatttcggtttcggccgccaggcgcacggctacccagacctgagcaccacctactgcggctcggccgcgtatgcgtcacctgaggtgctcctgggcatcccctacgaccctaagaaatacgacgtgtggagcctgggcgttgtgctctacgtcatggtcaccgggtgcatgcccttcgacgactcggacattgctggcctaccccggcgccagaagcgcggcgtgctctaccctgacggcctcgaactgtcggaacgatgcaaatccttgatcgccgagctgctacagttcagcccgtctgcccgtccctcggcgggccaggtggcgcgcaatggctggctgcgggccggggactccggctaggagccggggttctcgctgttcccgccgcaccaggcacagagccagggcggcgaacgcgcgaagggcgagcttcgggaaacccgcgaagtcgccgcgcgccactgcgcacgcgtcctgtcccttccccttcccccacggcggcggccctctgaggcctgctgttgggttctgattcctcctgcacaaactgattgctggagagcgcatgcgcatcctcgatttctggcgagaaggccccgggaggggcgcaaagagaagagagagaagcattgcgcctgcgcagaatgagctgttgcacggtgggttgccatgctacagagaaaccgtgggccagccgtgcacgctaagttccaggttggaacctgcaataaattcgctcttccttgcaaaaaaaaaaaaaaaaaaaaaaaaa

SEQ ID NO: 36 - DNA, 395 bp, mus musculus
tcgagttcatcgaagtgtgcaatgggaagctgtacatcgtgatggaggcagcagccaccgacctgctgcaggcagtgcagcgcaacgggcgcatcccggggtcgcaggcgcgcgaactcttctcgcagatcgcgggcgccgtgcgctacctgcacgaccaccacctggtgcaccgcgacctcaagtgcgaaaacgtgctgctaagccctgacgagcgccgcgtcaagatcacggatttcggtttcggccgccaggcgcacggctacccagacctgagcaccacctactgcggctcggccgcgtatgcgtcacctgaggtgctcctgggcatcccctacgaccctaagaaatacgacgtgtggagcctgggcgttgtgctctacgtcatggtcacc

SEQ ID NO: 37 - DNA, 20 bp, mus musculus
tcgagttcatcgaagtgtgc

SEQ ID NO: 38 - DNA, 20 bp, mus musculus
ggtgaccatgacgtagagca

SEQ ID NO: 39 - DNA, 1812 bp, homo sapien
acccccacaccatggcgagcgtggtggtgaagacgatctggcagtccaaagagatccatgaggccggggacacccccacgggggtggagagctgctcccagctagtcccagaggctccccggagggtgaccagccgggccaaggggatcccgaagaaaaagaaggccgtgtcgttccacggggtggagccccagatgtcccatcagcccatgcactggtgcctgaacctcaaacggtcctcggcctgcaccaacgtgtcactgctcaacctggccgccatggagcccactgactccacggggacagactccacagtggaagacctcagcggccaactcacactggctgggccccctgcctcccctaccctaccctgggatccggatgacgcagacatcacggaaatcctgagtggggtcaacagtggattggtccgcgccaaagactccatcaccagcttgaaggaaaagaccaaccgggttaaccagcacgtgcagtctctgcagagcgagtgttctgtgctgagcgagaatctggagagaaggcggcaagaggcagaagagttggaggggtactgcattcaactcaaggagaactgctggaaggtgacccggtctgtggaagatgctgaaatcaaaaccaacgtcttgaagcagaattctgccctgctggaggagaagctgcgctacctccagcagcagctgcaggatgagacgccgcgacggcaggaggccgagctgcaggagccggaggagaagcaggagccggaggagaagcaggagccggaggagaagcagaagccggaggctggcctctcctggaacagcctgggccccgccgccacgtcccagggctgccccggcccgccagggagtcccgacaaaccctcgcggccacacggcctggtccccgcaggctggggaatggggcctcgggctggcgagggcccctacgtgagcgagcaggaattgcagaagctgttcaccggcatcgaagagctgaggagagaggtgtcctcactgaccgcccggtggcatcaggaggagggggcggtgcaggaagccctgcggctgctcgggggcctgggcggcagggtcgacggcttcctaggccagtgggagcgggcacagcgcgaacaggcacagacggcgcgggacttgcaggagctgcgaggtcgggcggatgagctgtgcaccatggtggagcggtcagcagtgtctgtggcttcactgaggagcgaactggaggggctgggcccactgaaacccattctggaggagttcgggcggcaatttcagaactctcgaagagggcctgacctctccatgaacctggatcggtcccaccaaggcaactgtgcccgctgtgccagccaggggtcgcagttgtctacggagtccctgcagcagctgctggaccgagcactgacctcactagtggacgaggtgaagcagaggggcctgactcctgcctgtcccagctgtcagaggctacacaagaagattctggagctggagcgccaggccttagccaaacacgtcagggcagaggccctgagctccacccttcggctggcccaagacgaggccctgcgggccaagaacctactgctgacagacaagatgaagccagaggagaagatggccactctggaccatctacacttgaagatgtgctccctccacgatcatctcagcaacctgccacttgaggggtccacgggaacaatggggggaggcagcagtgcaggaacccccccaaaacaggggggctcagcccctgaacaataaatggcctctcatgctagcatga

SEQ ID NO: 40 - protein, 592 aa, homo sapien
MASVVVKTIWQSKEIHEAGDTPTGVESCSQLVPEAPRRVTSRAKGIPKKKKAVSFHGVEPQMSHQPMHWCLNLKRSSACTNVSLLNLAAMEPTDSTGTDSTVEDLSGQLTLAGPPASPTLPWDPDDADITEILSGVNSGLVRAKDSITSLKEKTNRVNQHVQSLQSECSVLSENLERRRQEAEELEGYCIQLKENCWKVTRSVEDAEIKTNVLKQNSALLEEKLRYLQQQLQDETPRRQEAELQEPEEKQEPEEKQEPEEKQKPEAGLSWNSLGPAATSQGCPGPPGSPDKPSRPHGLVPAGWGMGPRAGEGPYVSEQELQKLFTGIEELRREVSSLTARWHQEEGAVQEALRLLGGLGGRVDGFLGQWERAQREQAQTARDLQELRGRADELCTMVERSAVSVASLRSELEGLGPLKPILEEFGRQFQNSRRGPDLSMNLDRSHQGNCARCASQGSQLSTESLQQLLDRALTSLVDEVKQRGLTPACPSCQRLHKKILELERQALAKHVRAEALSSTLRLAQDEALRAKNLLLTDKMKPEEKMATLDHLHLKMCSLHDHLSNLPLEGSTGTMGGGSSAGTPPKQGGSAPEQ

SEQ ID NO: 41 - protein, 526 aa, mus musculus
MASVVVKTIWQSKEIHEAGDPPAGVESRAQLVPEAPGGVTSPAKGITKKKKAVSFHGVEPRMSHEPMHWCLNLKRSSACTNVSLLNLAAVEPDSSGTDSTTEDSGPLALPGTPASPTTPWAPEDPDITELLSGVNSGLVRAKDSITSLKEKTTRVNQHVQTLQSECSVLSENLERRRQEAEELEGYCSQLKGPRPDVLTQENCRKVTRSVEDAEIKTNVLKQNSALLEEKLRYLQQQLQDETPRRQEAELQELEQKLEAGLSRHGLSPATPIQGCSGPPGSPEEPPRQRGLSFSGWGMAVRTGEGPSLSEQELQKVSTGLEELRREVSSLAARWHQEEGAVQEPLRLLGGLGGRLDGFLGQWERAQREQAQSARGLQELRARADELCTMVERSAVSVASLRSELEALGPVKPILEELGRQLQNSRRGADHVLNLDRSAQGPCARCASQGQQLSTESLQQLLERALTPLVDEVKQKGLAPASPSCQRLHKKILELERQALAKHVRAEALSSTFGWPKTRPFGPRTYC

SEQ ID NO: 42 - DNA, 1814 bp, mus musculus
acctgggagcaggccccccgcaccatggcaagcgtggtggtgaagacaatctggcaatccaaagagatccacgaagcgggggacccacctgcgggggtagaaagccgtgcccagctggtccccgaggctcccgggggggtgaccagccctgccaaagggataacgaaaaaaaagaaggctgtgtccttccatggggtggagccccggatgtcccacgagccgatgcactggtgcctgaacctcaagcggtcctctgcctgcaccaacgtgtccttgctcaacctggctgccgtggagcccgactcctcgggcacagactcgaccacagaggacagtggtccactggcactgccagggacacctgcttcccctacaacaccctgggctccagaggaccctgacatcacagaactactgagtggggtcaacagtggattggtacgtgccaaagactccatcaccagcttgaaggaaaagaccacgcgggttaatcagcacgttcagactctgcagagcgagtgctctgtgctgagtgagaatctggaaagaagacggcaggaggcagaagagttggaggggtactgcagtcagttgaagggcccccgccctgatgtcctgacccaggagaactgccgcaaggtgacccgttcagtggaagacgctgaaatcaaaaccaatgtcctgaagcagaactctgccttgctggaggagaagctaagatacctccagcagcaactgcaggatgagacgccccggagacaggaggccgagttgcaggagttggagcaaaaactggaggctggcctctcccgacatggtctgagccctgccactcccattcagggctgctcgggtcctcctggcagccccgaggaacccccgcggcagcgaggcctgtccttcagtggctggggcatggcagtccgcacaggggagggaccctcgctgagcgagcaggagttgcagaaggtctccaccggcctggaggagctgaggagggaggtgtcctcgctggcagcccggtggcatcaggaggagggggcagtgcaggagcccctgaggttgctgggaggccttggcggccgtctggatggcttcctgggccagtgggagcgcgcgcagcgggaacaggcacagtccgcaaggggcttgcaggagctgcgagcacgagcagatgagttgtgcactatggtggagaggtcagcagtgtctgtcgcctcactgaggagtgaactggaggcactgggcccagtaaaacccattctggaggagctgggacggcaacttcagaactcccggaggggagctgaccatgtcttgaacttggatcggtctgcccaaggcccctgtgcgcgctgtgccagccaggggcagcagttatccacggagtccctgcagcagctgctggaacgtgcgctgaccccgctggtggatgaggtgaagcagaagggtctggctcctgccagccccagttgccagaggctacacaagaagattctggagctggagcgccaggccttggccaaacacgtcagggcagaggccctgagctccaccttcggttggcccaagacgaggccgttcgggccaagaacctactgctgacggacaagatgaagccggaggagaaggtggccactttggactatatgcatttgaagatgtgctccctccacgaccaactcagccacctgccacttgaggggtccacggggcaatggggggaggaagcaatggaggggctccccctaaacgtgggagtccaggctctgaacaataaatggcctctcatgctggcatgaaaaaaaaaaaaa

(19) United States
(12) Patent Application Publication
(10) Pub. No.: US 2008/0274117 A1
(43) Pub. Date: Nov. 6, 2008

(54) Validation of Tssk Family Members and Tsks as Male Contraceptive Targets

(76) Inventors: John C. Herr, Charlottesville, VA (US); Bingfang Xu, Charlottesville, VA (US); Zhonglin Hao, Gray, GA (US)

Correspondence Address: UNIVERSITY OF VIRGINIA PATENT FOUNDATION, 250 WEST MAIN STREET, SUITE 300, CHARLOTTESVILLE, VA, 22902, US

(21) Appl. No.: 11/664,213

(22) PCT Filed: Sep. 30, 2005
(86) PCT No.: PCT/US05/35029

§ 371 Date: Jul. 23, 2008

Related U.S. Application Data
(60) Provisional application No. 60/614,647, filed on Sep. 30, 2004.

(51) Int. Cl. A61K 39/395; A61K 38/16; C07H 21/00; C12N 5/10; A61P 15/16

(52) U.S. Cl. 424/141.1; 424/130.1; 514/12; 536/23.1; 435/366

(57) ABSTRACT

The present invention relates to a family of testis specific kinases (the TSSK family) and a substrate (TSKS) of TSSK, nucleic acid sequences encoding those kinases and substrate, and antibodies and antagonists against the kinases and substrate, methods of regulating these proteins, and their use as contraceptive targets.

### CROSS REFERENCE TO RELATED APPLICATIONS

This application claims priority under 35 U.S.C. § 119(e) to U.S. Provisional Application Ser. No. 60/614,647, filed Sep. 30, 2004, the disclosure of which is incorporated herein by reference.

### STATEMENT REGARDING FEDERALLY SPONSORED RESEARCH OR DEVELOPMENT

This invention was made with United States Government support under Grant Nos. HD 38082, and U54 29099, awarded by the National Institutes of Health. The United States Government may have certain rights in the invention.

### BACKGROUND

Spermatogenesis, the process in which functional sperm cells are produced in the testis, involves specific interaction between the developing germ cells and their supporting Sertoli cells as well as hormonal regulation by the androgen-producing Leydig cells. The general organization of spermatogenesis is essentially the same in all mammals and can be divided into three distinct phases: 1) The initial phase is the proliferative or spermatogonial phase during which spermatogonia undergo mitotic division and generate a pool of spermatocytes; 2) the meiotic phase, that yields the haploid spermatids; and 3) spermiogenesis whereby each round spermatid differentiates into a spermatozoon. Although the molecular mechanisms regulating the first two phases have been relatively well characterized, the molecular basis of spermiogenesis is largely unknown.

Mammalian spermiogenesis, the postmeiotic phase of spermatogenesis, is characterized by dramatic morphological changes that occur in the haploid spermatid. Some of these changes include the formation of the acrosome and its contents, the condensation and reorganization of the chromatin, the elongation and species-specific reshaping of the cell, and the assembly of the flagellum. These events result from changes in both gene transcription and protein translation that occurs during this developmental period. Some of the proteins translated in the haploid spermatid will remain in the morphologically mature sperm after it leaves the testis. Taking this into consideration, proteins that are synthesized during spermatogenesis might be necessary for spermatid differentiation and/or for sperm function during fertilization.

One aspect of the present invention relates to signaling events in mammalian sperm that regulate the functions of this highly differentiated cell. More particularly, in one embodiment the invention relates to signal transduction that modulates the acquisition of sperm fertilizing capacity. After ejaculation, sperm are able to move actively but lack fertilizing competence. They acquire the ability to fertilize in the female genital tract in a time-dependent process called capacitation. Capacitation has been demonstrated to be accompanied by phosphorylation of several proteins on both serine/threonine and tyrosine residues, and that protein tyrosine phosphorylation is regulated downstream by a cAMP/PKA pathway that involves the crosstalk between these two signaling pathways. With the exception of PKA, the other kinase(s) involved in the regulation of capacitation are still unknown.

Examples of testis-specific kinases include the recently described mouse genes, tssk 1, 2 and 3 (Bielke et al., 1994, Gene 139, 235-9; Kueng et al, 1997, J Cell Biol 139, 1851-9; Zuercher et al., 2000, Mech Dev 93, 175-7). Using a combination of two yeast hybrid technology and coimmunoprecipitation, Kueng et al. (1997) found that mouse tssk 1 and 2 bind and phosphorylate a protein of 54 Kda. That 54 Kda protein represents the tssk substrate, designated as tsks. The tsks protein is also testis-abundant and its developmental expression suggests that it is postmeiotically expressed in germ cells. The mouse cDNA sequence of the tssk substrate was previously reported (Kueng et al., 1997) and was used to search the EST data base. A human EST homologue AL041339 was found and used to generate sense and antisense primers for obtaining the full length clone by 5 and 3 RACE using human testis marathon ready cDNA (Clontech, Inc.).

The function of the tssk kinase family is largely unknown. However, since the members of this family are expressed postmeiotically during spermiogenesis, it is hypothesized that they have a role in germ cell differentiation, or later on in sperm function.

Spiridonov et al. have demonstrated that small serine/threonine protein kinase “SSTK”, which is expressed in elongating spermatids and specifically phosphorylates histones H1, H2A, H2AX, and H3, is essential for male fertility (Spiridonov et al., 2005, Molecular and Cellular Biology, 25:10:4250-4261). Targeted deletion of the SSTK gene in mice resulted in male sterility due to profound impairment in motility and morphology of spermatozoa (Spiridonov et al., 2005, Molecular and Cellular Biology, 25:10:4250-4261).

Despite the availability of a range of contraceptive methods, over 50% of pregnancies are unintended worldwide and in the United States. Thus, there is a critical need for contraception that better fits the diverse needs of women and men and takes into consideration different ethnic, cultural, and religious values. Except for the use of condoms or vasectomy, the availability of contraceptive methods for men is very limited.

There is a long felt need in the art to identify proteins involved in fertilization and methods to regulate this process. The present invention satisfies these needs.

### SUMMARY OF THE INVENTION

The present invention is directed to a family of sperm or testis specific kinases (tssk) genes, their respective encoded proteins, and their role in fertility. The present invention is also directed to the protein substrates of sperm specific kinases. More particularly, the present invention is directed to human kinases 1, 2, 3, and 4 (tssks) and the tssk kinase substrate tsks.

The present invention is also directed to compounds which are antagonists of tssks and tsks. Antagonists of tssk activity, interaction of tssk with tsks, and tsks activity, are anticipated to have utility as contraceptive agents. In one aspect, the invention is directed to methods of contraception encompassing blocking tssk activity. In another aspect, invention is directed to methods of contraception encompassing blocking tsks activity.

The present invention is also directed to methods of decreasing the synthesis, production, accumulation, and function of tssks and tsks.

In one embodiment, the tssk proteins are selected from the group consisting of proteins comprising amino acid sequences selected from the group consisting of human tssk1 (SEQ ID NO:4), tssk2 (SEQ ID NO:5), tssk3 (SEQ ID NO:6), and tssk4 (SEQ ID NO:20), or biologically active fragments or homologs thereof.

In one embodiment, the human tsks protein comprises the sequence SEQ ID NO:40, or biologically active fragments or homologs thereof. In one aspect, SEQ ID NO:40 is encoded by a nucleic acid having the sequence of SEQ ID NO:39.

In one embodiment, the invention provides methods of inhibiting fertilization, comprising contacting a sperm with an inhibitor of a tssk or a tsks. In another embodiment, the invention provides a method of decreasing fertility in a subject, comprising administering an effective amount of an inhibitor of tssk or tsks. In one embodiment, the invention provides a contraceptive vaccine, comprising administering one or more tssk and/or tsks proteins, or immunogenic fragments thereof.

The invention provides methods for preparing knockout constructs of tssks or tsks. In one aspect, the constructs are useful for preparing knockout mice. In one aspect, the construct represents a double knockout.

The invention provides kits for inhibiting tssk and tsks function.

### BRIEF DESCRIPTION OF THE DRAWINGS

FIG. 1 represents a sequence alignment comparison of TSSK1, TSSK2, TSSK3, and TSSK4 amino acid sequences. Amino acids found in at least two of the four aligned sequences are shaded to show identity. The highest homology is found between TSSK1 and TSSK2 (83% in the kinase domain and 72% across the entire ORF). TSSK3 is 47.5% and 49% identical to TSSK1 and TSSK2 respectively. TSSK4 is 49% identical to TSSK3. The highly conserved signature sequence that fits the consensus “DLKXXN” for serine/threonine kinases is underlined. The 12 kinase subdomains are marked above the alignment with roman numerals.

FIG. 2 is a schematic representation of the domain structure of the mammalian TSSK subfamily. TSSK1-4 are predicted to be active kinases, the kinase domains compose the major part of the TSSKs. TSSK1 and TSSK are longer at their carboxyl termini than TSSK 3 and TSSK4. The predicted ATP binding sites are marked.

FIG. 3 is a schematic representation of the Phylogenetic relationship of TSSKs with other kinases, and the relationship between the TSSK family members. TSSK 1 and TSSK2 form a subgroup within the TSSK family.

FIG. 4, comprising FIGS. 4A and 4B, represents Northern (4A) and dot blot (4B) analyses of human TSSK1 expression. FIG. 4A represents the results of a Northern blot analysis of TSSK1 expression in eight human tissues. Beta actin serves as a lane loading control. TSSK1 transcripts were only located in testis, and the lower band of slightly faster migration rate in the Testis may be a TSSK-pseudogene mRNA. FIG. 4B represents a dot blot analysis of TSSK1 expression in 72 human tissues, under stringent wash conditions.

FIG. 5, comprising FIGS. 5A and 5B, represents Northern (5A) and dot blot (5B) analyses of human TSSK2 expression. FIG. 5A represents the results of a Northern blot analysis of TSSK2 expression in eight human tissues. Beta actin serves as a lane loading control. FIG. 5B represents a dot blot analysis of TSSK2 expression in 72 human tissues. TSSK2 transcripts were only located in testis.

FIG. 6, comprising FIGS. 6A and 6B, represents Northern blot analyses of TSSK3 (FIG. 6A) expression in eight human tissues and TSSK4 (FIG. 6B) expression in eight mouse tissues. Each was only expressed in testis. It can be seen in FIG. 6A that TSSK3 mRNA may have multiple splice sites.

FIG. 7, comprising FIGS. 7A, 7B, 7C, and 7D, graphically represents Real-time PCR analysis of TSSK1 (7A), TSSK2 (7B), TSSK3 (7C), and TSSK4 (7D) expression in fifteen human tissues. TSSK 3 and 4 transcripts were detected only in testis, while TSSK 1 was also detectable in pancreas and TSSK2 was also detectable in heart, brain, placenta, and liver.

FIG. 8 graphically represents real-time PCR quantitation of human TSSK1-4 relative expression levels in Testis. mRNA abundance is shown in log scale. TSSK1 and 2 transcripts are approximately 10 times more abundant than TSSK 3 and 4 in human testis. Experiments are shown in triplicate (black, light gray, and dark gray bars).

FIG. 9 schematically demonstrates the strategy of double knock out of TSSK1 and TSSK2 using a single construct.

FIG. 10, comprising panels 1 through 6 (FIGS. 10A through 10B), represents photomicrographic images of in situ analyses of TSSK2 mRNA expression in adult mouse testis, indicating post-meiotic expression and sperm equatorial segment localization of TSSK2. In situ hybridization of TSSK2 transcripts was carried out using radiolabeled mouse TSSK2 cRNA. The low-magnification (×100) views of seminiferous tubules hybridized with sense TSSK2 (10A) or antisense TSSK (10B) are shown in dark field, the higher magnification (×200, ×400) views of seminiferous tubules hybridized with antisense TSSK2 are also shown in both bright-field (10C and 10D) and dark field (10E and 10F). TSSK2 transcripts are expressed mainly in the post-meiotic spermatids, while the labeling on the primary spermatocytes is not much greater than the background.

FIG. 11, comprising panels 1 through 6 (FIGS. 11A through 11B), represents photomicrographic images of in situ analyses of TSSK4 mRNA expression in adult mouse testis, indicating post-meiotic expression and sperm equatorial segment localization of TSSK4. In situ hybridization of TSSK4 transcripts was carried out using radiolabeled mouse TSSK4 cRNA. The low-magnification (×100) views of seminiferous tubules hybridized with sense TSSK4 (11A) or antisense TSSK4 (11B) are shown in dark field, the higher magnification (×200, ×400) views of seminiferous tubules hybridized with antisense TSSK4 are shown in both bright-field (11C and 11D), and dark field (11E and 11F). TSSK4 transcripts are expressed mainly in the post-meiotic spermatids.

FIG. 12 represents images of a Western blot analysis of TSSK2 expression in human testis and sperm.

FIG. 13 schematically represents the 3D structure of mouse TSSK4 modeled by computer. A peptide (RRAPPDFVNKFLPRE) identical in mouse and human TSSK4 proteins was used to generate antibody against TSSK4. Mouse TSSK4 structure was modeled to show that this peptide is on the surface of the molecule after folding.

FIG. 14, comprising FIGS. 14A and 14B, represents photomicrographic images of immunofluorescent localization (14A) of TSSK2 in ejaculated sperm. TSSK2 is localized weakly to the acrosomal cap and more intensely to the equatorial segment. Its localization to the equatorial segment of the sperm suggests a role for TSSK2 during fertilization.

FIG. 15 represents a Western blot analysis of TSSK4 expression in mouse testis, mouse sperm, and human sperm. A single band with the predicted molecular size of TSSK4 was detected in mouse sperm protein extracts using rabbit TSSK4 anti-serum, while a lower molecular weight TSSK4 protein (indicated by “?”), a likely proteolytic product, was detected in human sperm protein extracts. The higher molecular weight band in the protein extracts from mouse testis may be a TSSK4 dimer.

FIG. 16, comprising six panels (FIGS. 16A through 16F), represents photomicrographic images of the immunofluorescent localization of TSSK4 in mouse and human sperm. In the upper two panels (FIGS. 16A and 16B), immunofluorescent staining of human sperm was performed using affinity purified anti-TSSK4 antibody and secondary antibody alone. In the lower four panels (FIGS. 16C, D, E, and F) immunofluorescent staining of mouse sperm was performed using affinity purified anti-TSSK4 antibody (16C and 16D) and normal rabbit IgG (16E and 16F). TSSK4 is localized to the equatorial segment in both mouse and human sperm. Its localization to the equatorial segment of the sperm might indicate a role for TSSK4 during fertilization.

FIG. 17, comprising FIGS. 17A and 17B, represents images of Northern (17A; eight human tissues) and dot (17B; 72 human tissues) blot analyses of TSKS expression, demonstrating testis specific and post-meiotic expression of human TSKS. TSKS transcripts were only detected in testis.

FIG. 18 represents an image of a Western blot analysis of TSKS expression. Human TSKS protein was found in human testis but was not detected in mature spermatozoa.

FIG. 19, comprising six panels, represents photomicrographic images of the analysis of TSKS mRNA expression in adult mouse testis. In situ hybridization of TSKS transcripts was carried out using radiolabeled mouse TSKS cRNA. The low-magnification (×100) views of seminiferous tubules hybridized with sense TSKS (1) or antisense TSKS (2) are shown in dark field, the higher magnification (×200, ×400) views of seminiferous tubules hybridized with antisense TSKS are also shown in both bright-field (3,5), and dark field (4,6). TSKS transcripts were expressed mainly in the post-meiotic spermatids, and the labeling of the primary spermatocytes or spermatogonia is not much greater than the background.

FIG. 20, comprising FIGS. 20A, 20B, and 20C, represents photomicrographic images of immunofluorescent staining of TSKS in disassociated cells from human testis. Human TSKS, red signal, was detected in round spermatids (A) as small immunofluorescent dots detached from the nuclei [blue, DAPI stained] at the acrosomal poles. In early and late elongating spermatids (B), TSKS immunofluorescence was similarly adjacent to the acrosomal pole and reached its peak intensity and size, suggesting association of TSKS with the Golgi apparatus. TSKS immunofluorescence was virtually absent in mature testicular sperm (C).

FIG. 21, comprising FIGS. 21A (left 4 panels) and 21B (right panels/gels), represents photomicrographic images of the results of studies on the interaction of human TSSK2 and TSKS in a yeast two hybrid system (21A) and the confirmation of hybrid protein co-expression by western blotting (21B).

FIG. 21A depicts interaction of TSSK2 and TSKS in a yeast two hybrid system. Yeast host strain AH109 was transformed with either a pair of plasmids or a single plasmid as follows: pGAD, pGBK-TSSK2 (1); pGAD-TSKS, pGBK-TSSK2 (2); pGAD, pGBKp53 (3); pGAD-lgT, pGBK-p53 (4); pGAD (5); pGAD-TSKS (6); and pGAD-LgT (7). The transformants were streaked on complete drop-out media lacking both leucine and tryptophan (SCM-L-T), leucine (SCM-L), both leucine and histidine (SCM-L-H), or leucine, tryptophan and histidine (SCM-L-T-H) to test for histidine prototrophy. The p53 and the SV40 large T antigen controls (4) as well as TSSK2 and TSKS (2) interacted in the yeast two hybrid system.

FIG. 21B represents confirmation of hybrid protein co-expression by western blotting. ORFs of TSSK2 and TSKS were fused with GalDBD and GalAD respectively and co-transformed into AH109 host strain. The strains were tested for expression of DBD-TSSK (myc tagged), AD-TSKS (HA tagged). DBD-P53 (myc tagged) and GAD-LgT (HA tagged) were used as positive controls. Both TSSK and TSKS were co-expressed in AH109 validating this model for further studies of binding interaction.

FIG. 22 graphically illustrates a reporter gene activity assay (α-galactosidase) of a yeast strain harboring each pair of hybrid proteins, as measured by the quantitation of the binding strength between TSSK2 and TSKS. Alpha-galactosidase activity was measured at OD 410 by incubating the culture supernatants of AH109 harboring each pair of plasmids as indicated using r-nitrophenyl-a-D-Galactopyranoside as a substrate. The activities were expressed as arbitrary units after calibration the optical density of the culture supernatants. TSKS binding strength with TSSK2 was 12 times stronger than the negative control while being 1.5 times stronger than the positive control interaction between p53 and LgT.

FIG. 23 represents an image of an electrophoretic analysis demonstrating co-immunoprecipitation of human TSSK2 with human TSKS in vitro. TSSK2 (myc tagged) and TSKS (HA tagged) were co-translated in a Promega in-vitro translation kit. The mixture was immunoprecipitated with either agarose beads coupled to monoclonal antibody against myc (mice) or HA (rat). Immunoprecipitations were separated with SDS-PAGE and blotted with either anti-HA (left) or anti-myc (right): 1. In vitro translation mix; 2. In vitro translation control; 3. IP (anti-HA); 4. HA-IP control; 5. IP (anti-Myc); and 6. Myc-IP control. Arrows indicate that using either anti-myc or anti-HA, TSSK and TSKS co-immunoprecipitate, confirming their interactions.

FIG. 24, comprising FIGS. 24A and 24B, represents an analysis of the essential interacting domains of TSKS required for interaction with TSSK. FIG. 24A represents a schematic drawing of TSKS ORF (top panel). Two putative coiled-coil domains [CC1, CC2] are filled. The six amino-acid repeats are hatched, and the amino terminus nuclear localization signal is indicated in gray. A total of 8 deletion mutants of TSKS were created and fused with the Gal4 activation domain in the pGAD two hybrid vector. Fusion proteins of each mutant and the Gal-AD were co-transformed with Gal-DBDTSSK into the yeast host strain AH109. FIG. 24B represents culture supernatants of each pair which were assayed for alpha-galactosidase activity and the activity measured for each mutant is expressed as percentage of full length TSKS. The corresponding deletion mutant is indicated on the ordinate. Deletion of the first 48 amino acids reduces TSKS binding to 5%, while deletion of the first 147 amino acids abolishes TSKS binding. Deletion of the carboxyl terminus and second coiled-coil region reduces interaction by 75%.

FIG. 25, comprising FIGS. 25A and 25B, represents images of studies of the phosphorylation of mouse TSKS in vitro. Immunoprecipitation of TSKS from mouse testis using rat antiserum against TSKS. Precleared mouse testis extracts were immunoprecipitated with either rat normal serum (control IP), or rat anti-TSKS serum (TSKS IP), the immunoprecipitated complexes were separated on 1D (FIG. 25A) and 2D (FIG. 25B) gels, and subjected to silver staining and immunoblotting with anti-TSKS antibody. This demonstrated that the rat anti-human TSKS serum was capable of immunoprecipitating mouse TSKS.

FIG. 26 represent an image of an electrophoretic analysis of in vitor phosphorylation of TSKS. Autoradiograph of immune-complexes immunoprecipitated with either anti-TSKS (TSKS IP) or rat normal serum (control IP) and subsequently incubated with γP32 ATP in an in vitro kinase assay. A common kinase, casein kinase 2 (CKII) and several common kinase substrates including casein, maltose binding protein (MBP) and histone 3 were added to the reactions as the positive control. With anti-TSKS sera TSKS was strongly phosphorylated as well as a 42 KD protein, while no proteins were phosphorylated with control normal rat sera. The 42 KD protein may be autophosphorylation of TSSK1 and/or TSSK. Addition of CKII did not result in any additional phosphorylation indicating either phosphorylation of TSKS and TSSK is saturated or they are not substrates of CKII. Casein, MBP and histone3 were also phosphorylated by the kinases in the precipitated complex, whereas these proteins were not phosphorylated in the controls immunoprecipitated by normal sera. This kinase assay provides a high throughput screen for inhibitors of TSKS phosphorylation as well as demonstrating that casein, MBP and histone 3 may be used as alternative substrates for TSSK1 and TSSK.

### DETAILED DESCRIPTION OF THE INVENTION

### Abbreviations

Bovine serum albumin (BSA)

capacitating conditions or capacitated sperm (CAP)

caveolin 1 (CAV1)

detergent-resistant membrane (DRM)

Hexokinase I (HK1)

High density lipoprotein (HDL)

Horseradish peroxidase (HRP)

human sperm PRV-like protein (hsPRV)

mouse sperm PRV-like protein (msPRV)

non-capacitating conditions or non-capacitated sperm (NON)

Polycythemia rubra vera (PRV)

room temperature (RT)

sperm PRV-like protein (sPRV)

Tris-Buffered Saline, pH 7.3, 0.1% Tween 20 (TBST)

TSKS— substrate of testis/sperm specific kinase (also referred to as tsks)

TSSK—testis/sperm specific serine kinase

Whitten's—HEPES buffered (WH)

### DEFINITIONS

In describing and claiming the invention, the following terminology will be used in accordance with the definitions set forth below.

The articles “a” and “an” are used herein to refer to one or to more than one (i.e., to at least one) of the grammatical object of the article. By way of example, “an element” means one element or more than one element.

A disease or disorder is “alleviated” if the severity of a symptom of the disease or disorder, the frequency with which such a symptom is experienced by a patient, or both, are reduced.

As used herein, “amino acids” are represented by the full name thereof, by the three letter code corresponding thereto, or by the one-letter code corresponding thereto, as indicated in the following table:

| Full Name | Three-Letter Code | One-Letter Code |
|---|---|---|
| Aspartic Acid | Asp | D |
| Glutamic Acid | Glu | E |
| Lysine | Lys | K |
| Arginine | Arg | R |
| Histidine | His | H |
| Tyrosine | Tyr | Y |
| Cysteine | Cys | C |
| Asparagine | Asn | N |
| Glutamine | Gln | Q |
| Serine | Ser | S |
| Threonine | Thr | T |
| Glycine | Gly | G |
| Alanine | Ala | A |
| Valine | Val | V |
| Leucine | Leu | L |
| Isoleucine | Ile | I |
| Methionine | Met | M |
| Proline | Pro | P |
| Phenylalanine | Phe | F |
| Tryptophan | Trp | W |

The expression “amino acid” as used herein is meant to include both natural and synthetic amino acids, and both D and L amino acids. “Standard amino acid” means any of the twenty standard L-amino acids commonly found in naturally occurring peptides. “Nonstandard amino acid residue” means any amino acid, other than the standard amino acids, regardless of whether it is prepared synthetically or derived from a natural source. As used herein, “synthetic amino acid” also encompasses chemically modified amino acids, including but not limited to salts, amino acid derivatives (such as amides), and substitutions. Amino acids contained within the peptides of the present invention, and particularly at the carboxy- or amino-terminus, can be modified by methylation, amidation, acetylation or substitution with other chemical groups which can change the peptide's circulating half-life without adversely affecting their activity. Additionally, a disulfide linkage may be present or absent in the peptides of the invention.

The term “amino acid” is used interchangeably with “amino acid residue,” and may refer to a free amino acid and to an amino acid residue of a peptide. It will be apparent from the context in which the term is used whether it refers to a free amino acid or a residue of a peptide.

Amino acids have the following general structure:

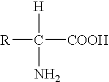

Amino acids may be classified into seven groups on the basis of the side chain R: (1) aliphatic side chains, (2) side chains containing a hydroxylic (OH) group, (3) side chains containing sulfur atoms, (4) side chains containing an acidic or amide group, (5) side chains containing a basic group, (6) side chains containing an aromatic ring, and (7) proline, an imino acid in which the side chain is fused to the amino group.

The nomenclature used to describe the peptide compounds of the present invention follows the conventional practice wherein the amino group is presented to the left and the carboxy group to the right of each amino acid residue. In the formulae representing selected specific embodiments of the present invention, the amino- and carboxy-terminal groups, although not specifically shown, will be understood to be in the form they would assume at physiologic pH values, unless otherwise specified.

As used herein, an “analog” of a chemical compound is a compound that, by way of example, resembles another in structure but is not necessarily an isomer (e.g., 5-fluorouracil is an analog of thymine).

The term “basic” or “positively charged” amino acid as used herein, refers to amino acids in which the R groups have a net positive charge at pH 7.0, and include, but are not limited to, the standard amino acids lysine, arginine, and histidine.

The term “antibody,” as used herein, refers to an immunoglobulin molecule which is able to specifically bind to a specific epitope on an antigen. Antibodies can be intact immunoglobulins derived from natural sources or from recombinant sources and can be immunoreactive portions of intact immunoglobulins. Antibodies are typically tetramers of immunoglobulin molecules. The antibodies in the present invention may exist in a variety of forms including, for example, polyclonal antibodies, monoclonal antibodies, Fv, Fab and F(ab)₂, as well as single chain antibodies and humanized antibodies.

As used herein, the term “antisense oligonucleotide” or antisense nucleic acid means a nucleic acid polymer, at least a portion of which is complementary to a nucleic acid which is present in a normal cell or in an affected cell. “Antisense” refers particularly to the nucleic acid sequence of the non-coding strand of a double stranded DNA molecule encoding a protein, or to a sequence which is substantially homologous to the non-coding strand. As defined herein, an antisense sequence is complementary to the sequence of a double stranded DNA molecule encoding a protein. It is not necessary that the antisense sequence be complementary solely to the coding portion of the coding strand of the DNA molecule. The antisense sequence may be complementary to regulatory sequences specified on the coding strand of a DNA molecule encoding a protein, which regulatory sequences control expression of the coding sequences. The antisense oligonucleotides of the invention include, but are not limited to, phosphorothioate oligonucleotides and other modifications of oligonucleotides.

As used herein, the term “antisense oligonucleotide” or antisense nucleic acid means a nucleic acid polymer, at least a portion of which is complementary to a nucleic acid which is present in a normal cell or in an affected cell. “Antisense” refers particularly to the nucleic acid sequence of the non-coding strand of a double stranded DNA molecule encoding a protein, or to a sequence which is substantially homologous to the non-coding strand. As defined herein, an antisense sequence is complementary to the sequence of a double stranded DNA molecule encoding a protein. It is not necessary that the antisense sequence be complementary solely to the coding portion of the coding strand of the DNA molecule. The antisense sequence may be complementary to regulatory sequences specified on the coding strand of a DNA molecule encoding a protein, which regulatory sequences control expression of the coding sequences. The antisense oligonucleotides of the invention include, but are not limited to, phosphorothioate oligonucleotides and other modifications of oligonucleotides.

As used herein, the terms “complementary” or “complementarity” are used in reference to polynucleotides (i.e., a sequence of nucleotides) related by the base-pairing rules. For example, for the sequence “A-G-T,” is complementary to the sequence “T-C-A.”

As used herein, the term “biologically active fragments” or “bioactive fragment” of the polypeptides encompasses natural or synthetic portions of the full-length protein that are capable of specific binding to their natural ligand or of performing the function of the protein.

“Complementary” refers to the broad concept of sequence complementarity between regions of two nucleic acid strands or between two regions of the same nucleic acid strand. It is known that an adenine residue of a first nucleic acid region is capable of forming specific hydrogen bonds (“base pairing”) with a residue of a second nucleic acid region which is antiparallel to the first region if the residue is thymine or uracil. Similarly, it is known that a cytosine residue of a first nucleic acid strand is capable of base pairing with a residue of a second nucleic acid strand which is antiparallel to the first strand if the residue is guanine. A first region of a nucleic acid is complementary to a second region of the same or a different nucleic acid if, when the two regions are arranged in an antiparallel fashion, at least one nucleotide residue of the first region is capable of base pairing with a residue of the second region. Preferably, the first region comprises a first portion and the second region comprises a second portion, whereby, when the first and second portions are arranged in an antiparallel fashion, at least about 50%, and preferably at least about 75%, at least about 90%, or at least about 95% of the nucleotide residues of the first portion are capable of base pairing with nucleotide residues in the second portion. More preferably, all nucleotide residues of the first portion are capable of base pairing with nucleotide residues in the second portion.

A “compound,” as used herein, refers to a polypeptide, an isolated nucleic acid, or other agent used in the method of the invention.

As used herein, the term “conservative amino acid substitution” is defined herein as an amino acid exchange within one of the following five groups:

I. Small aliphatic, nonpolar or slightly polar residues:
Ala, Ser, Thr, Pro, Gly;

II. Polar, negatively charged residues and their amides:
Asp, Asn, Glu, Gln;

III. Polar, positively charged residues:
H is, Arg, Lys;

IV. Large, aliphatic, nonpolar residues:
Met Leu, Ile, Val, Cys

V. Large, aromatic residues:
Phe, Tyr, Trp

A “control” cell, tissue, sample, or subject is a cell, tissue, sample, or subject of the same type as a test cell, tissue, sample, or subject. The control may, for example, be examined at precisely or nearly the same time the test cell, tissue, sample, or subject is examined. The control may also, for example, be examined at a time distant from the time at which the test cell, tissue, sample, or subject is examined, and the results of the examination of the control may be recorded so that the recorded results may be compared with results obtained by examination of a test cell, tissue, sample, or subject. The control may also be obtained from another source or similar source other than the test group or a test subject, where the test sample is obtained from a subject suspected of having a disease or disorder for which the test is being performed.

A “test” cell, tissue, sample, or subject is one being examined or treated.

A “pathoindicative” cell, tissue, or sample is one which, when present, is an indication that the animal in which the cell, tissue, or sample is located (or from which the tissue was obtained) is afflicted with a disease or disorder. By way of example, the presence of one or more breast cells in a lung tissue of an animal is an indication that the animal is afflicted with metastatic breast cancer.

A tissue “normally comprises” a cell if one or more of the cell are present in the tissue in an animal not afflicted with a disease or disorder.

A “compound,” as used herein, refers to any type of substance or agent that is commonly considered a drug, or a candidate for use as a drug, combinations, and mixtures of the above, as well as polypeptides and antibodies of the invention.

The use of the word “detect” and its grammatical variants is meant to refer to measurement of the species without quantification, whereas use of the word “determine” or “measure” with their grammatical variants are meant to refer to measurement of the species with quantification. The terms “detect” and “identify” are used interchangeably herein.

As used herein, a “detectable marker” or a “reporter molecule” is an atom or a molecule that permits the specific detection of a compound comprising the marker in the presence of similar compounds without a marker. Detectable markers or reporter molecules include, e.g., radioactive isotopes, antigenic determinants, enzymes, nucleic acids available for hybridization, chromophores, fluorophores, chemiluminescent molecules, electrochemically detectable molecules, and molecules that provide for altered fluorescence-polarization or altered light-scattering.

A “disease” is a state of health of an animal wherein the animal cannot maintain homeostasis, and wherein if the disease is not ameliorated then the animal's health continues to deteriorate.

In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

Unless otherwise specified, a “nucleotide sequence encoding an amino acid sequence” includes all nucleotide sequences that are degenerate versions of each other and that encode the same amino acid sequence. Nucleotide sequences that encode proteins and RNA may include introns.

An “enhancer” is a DNA regulatory element that can increase the efficiency of transcription, regardless of the distance or orientation of the enhancer relative to the start site of transcription.

A “fragment” or “segment” is a portion of an amino acid sequence, comprising at least one amino acid, or a portion of a nucleic acid sequence comprising at least one nucleotide. The terms “fragment” and “segment” are used interchangeably herein.

A “fragment” or “segment” is a portion of an amino acid sequence, comprising at least one amino acid, or a portion of a nucleic acid sequence comprising at least one nucleotide. The terms “fragment” and “segment” are used interchangeably herein.

As used herein, a “functional” biological molecule is a biological molecule in a form in which it exhibits a property or activity by which it is characterized. A functional enzyme, for example, is one which exhibits the characteristic catalytic activity by which the enzyme is characterized.

“Homologous” as used herein, refers to the subunit sequence similarity between two polymeric molecules, e.g., between two nucleic acid molecules, e.g., two DNA molecules or two RNA molecules, or between two polypeptide molecules. When a subunit position in both of the two molecules is occupied by the same monomeric subunit, e.g., if a position in each of two DNA molecules is occupied by adenine, then they are homologous at that position. The homology between two sequences is a direct function of the number of matching or homologous positions, e.g., if half (e.g., five positions in a polymer ten subunits in length) of the positions in two compound sequences are homologous then the two sequences are 50% homologous, if 90% of the positions, e.g., 9 of 10, are matched or homologous, the two sequences share 90% homology. By way of example, the DNA sequences 3′ATTGCC5′ and 3′TATGGC share 50% homology.

As used herein, “homology” is used synonymously with “identity.”

The determination of percent identity between two nucleotide or amino acid sequences can be accomplished using a mathematical algorithm. For example, a mathematical algorithm useful for comparing two sequences is the algorithm of Karlin and Altschul (1990, Proc. Natl. Acad. Sci. USA 87:2264-2268), modified as in Karlin and Altschul (1993, Proc. Natl. Acad. Sci. USA 90:5873-5877). This algorithm is incorporated into the NBLAST and XBLAST programs of Altschul, et al. (1990, J. Mol. Biol. 215:403-410), and can be accessed, for example at the National Center for Biotechnology Information (NCBI) world wide web site. BLAST nucleotide searches can be performed with the NBLAST program (designated “blastn” at the NCBI web site), using the following parameters: gap penalty=5; gap extension penalty=2; mismatch penalty=3; match reward=1; expectation value 10.0; and word size=11 to obtain nucleotide sequences homologous to a nucleic acid described herein. BLAST protein searches can be performed with the XBLAST program (designated “blastn” at the NCBI web site) or the NCBI “blastp” program, using the following parameters: expectation value 10.0, BLOSUM62 scoring matrix to obtain amino acid sequences homologous to a protein molecule described herein. To obtain gapped alignments for comparison purposes, Gapped BLAST can be utilized as described in Altschul et al. (1997, Nucleic Acids Res. 25:3389-3402). Alternatively, PSI-Blast or PHI-Blast can be used to perform an iterated search which detects distant relationships between molecules (Id.) and relationships between molecules which share a common pattern. When utilizing BLAST, Gapped BLAST, PSI-Blast, and PHI-Blast programs, the default parameters of the respective programs (e.g., XBLAST and NBLAST) can be used.

The percent identity between two sequences can be determined using techniques similar to those described above, with or without allowing gaps. In calculating percent identity, typically exact matches are counted.

The term “inhibit,” as used herein, refers to the ability of a compound of the invention to reduce or impede a described function, such as capacitation or fertilization. Preferably, inhibition is by at least 10%, more preferably by at least 25%, even more preferably by at least 50%, and most preferably, the function is inhibited by at least 75%.

As used herein “an inhibitor of tssk kinase activity” is intended to include any compound, composition or environmental factor that decreases overall tssk kinase activity without significantly impacting the activity of non-tssk kinases. Thus inhibitors include factors that decrease the specific activity of the kinase as well as factors that decrease the number of active kinase molecules available for the reaction (i.e. transcriptional and translational inhibitors for in vivo situations).

As used herein “an inhibitor of tsks activity” is intended to include any compound, composition, or environmental factor that decreases overall interaction of tsks with tssk kinases without significantly impacting the activity of non-tssk kinases, or inhibits the activity of tsks if phosphorylated by a tssk.

As used herin, “inhibiting tssk” or “inhibiting tsks” refers to any method or technique which inhibits tssk or tsks synthesis, production, formation, accumulation, or function, as well as methods of inhibiting the induction or stimulation of synthesis, formation, accumulation, or function or tssk or tsks. It also refers to any metabolic pathway which can regulate tssk or tsks function or regulation. Inhibition can be direct or indirect.

As used herein, an “instructional material” includes a publication, a recording, a diagram, or any other medium of expression which can be used to communicate the usefulness of the peptide of the invention in the kit for effecting alleviation of the various diseases or disorders recited herein. Optionally, or alternately, the instructional material may describe one or more methods of alleviating the diseases or disorders in a cell or a tissue of a mammal. The instructional material of the kit of the invention may, for example, be affixed to a container which contains the identified compound invention or be shipped together with a container which contains the identified compound. Alternatively, the instructional material may be shipped separately from the container with the intention that the instructional material and the compound be used cooperatively by the recipient.

An “isolated nucleic acid” refers to a nucleic acid segment or fragment which has been separated from sequences which flank it in a naturally occurring state, e.g., a DNA fragment which has been removed from the sequences which are normally adjacent to the fragment, e.g., the sequences adjacent to the fragment in a genome in which it naturally occurs. The term also applies to nucleic acids which have been substantially purified from other components which naturally accompany the nucleic acid, e.g., RNA or DNA or proteins, which naturally accompany it in the cell. The term therefore includes, for example, a recombinant DNA which is incorporated into a vector, into an autonomously replicating plasmid or virus, or into the genomic DNA of a prokaryote or eukaryote, or which exists as a separate molecule (e.g., as a cDNA or a genomic or cDNA fragment produced by PCR or restriction enzyme digestion) independent of other sequences. It also includes a recombinant DNA which is part of a hybrid gene encoding additional polypeptide sequence.

Unless otherwise specified, a “nucleotide sequence encoding an amino acid sequence” includes all nucleotide sequences that are degenerate versions of each other and that encode the same amino acid sequence. Nucleotide sequences that encode proteins and RNA may include introns.

As used herein, a “ligand” is a compound that specifically binds to a target compound. A ligand (e.g., an antibody) “specifically binds to” or “is specifically immunoreactive with” a compound when the ligand functions in a binding reaction which is determinative of the presence of the compound in a sample of heterogeneous compounds. Thus, under designated assay (e.g., immunoassay) conditions, the ligand binds preferentially to a particular compound and does not bind to a significant extent to other compounds present in the sample. For example, an antibody specifically binds under immunoassay conditions to an antigen bearing an epitope against which the antibody was raised. A variety of immunoassay formats may be used to select antibodies specifically immunoreactive with a particular antigen. For example, solid-phase ELISA immunoassays are routinely used to select monoclonal antibodies specifically immunoreactive with an antigen. See Harlow and Lane, 1988, Antibodies, A Laboratory Manual, Cold Spring Harbor Publications, New York, for a description of immunoassay formats and conditions that can be used to determine specific immunoreactivity.

As used herein, the term “linkage” refers to a connection between two groups. The connection can be either covalent or non-covalent, including but not limited to ionic bonds, hydrogen bonding, and hydrophobic/hydrophilic interactions.

As used herein, the term “linker” refers to a molecule that joins two other molecules either covalently or noncovalently, e.g., through ionic or hydrogen bonds or van der Waals interactions.

By “modulating fertility” is meant reducing or increasing fertility. For example, inhibiting fertilization is a means of modulating fertility.

By “nucleic acid” is meant any nucleic acid, whether composed of deoxyribonucleosides or ribonucleosides, and whether composed of phosphodiester linkages or modified linkages such as phosphotriester, phosphoramidate, siloxane, carbonate, carboxymethylester, acetamidate, carbamate, thioether, bridged phosphoramidate, bridged methylene phosphonate, bridged phosphoramidate, bridged phosphoramidate, bridged methylene phosphonate, phosphorothioate, methylphosphonate, phosphorodithioate, bridged phosphorothioate or sulfone linkages, and combinations of such linkages. The term nucleic acid also specifically includes nucleic acids composed of bases other than the five biologically occurring bases (adenine, guanine, thymine, cytosine and uracil). Conventional notation is used herein to describe polynucleotide sequences: the left-hand end of a single-stranded polynucleotide sequence is the 5′-end; the left-hand direction of a double-stranded polynucleotide sequence is referred to as the 5′-direction. The direction of 5′ to 3′ addition of nucleotides to nascent RNA transcripts is referred to as the transcription direction. The DNA strand having the same sequence as an mRNA is referred to as the “coding strand”; sequences on the DNA strand which are located 5′ to a reference point on the DNA are referred to as “upstream sequences”; sequences on the DNA strand which are 3′ to a reference point on the DNA are referred to as “downstream sequences.”

The term “oligonucleotide” typically refers to short polynucleotides, generally no greater than about 50 nucleotides. It will be understood that when a nucleotide sequence is represented by a DNA sequence (i.e., A, T, G, C), this also includes an RNA sequence (i.e., A, U, G, C) in which “U” replaces “T.”

“Operably linked” refers to a juxtaposition wherein the components are configured so as to perform their usual function. Thus, control sequences or promoters operably linked to a coding sequence are capable of effecting the expression of the coding sequence. By describing two polynucleotides as “operably linked” is meant that a single-stranded or double-stranded nucleic acid moiety comprises the two polynucleotides arranged within the nucleic acid moiety in such a manner that at least one of the two polynucleotides is able to exert a physiological effect by which it is characterized upon the other. By way of example, a promoter operably linked to the coding region of a gene is able to promote transcription of the coding region.

As used herein, a “peptide” encompasses a sequence of 2 or more amino acid residues wherein the amino acids are naturally occurring or synthetic (non-naturally occurring) amino acids covalently linked by peptide bonds. No limitation is placed on the number of amino acid residues which can comprise a protein's or peptide's sequence. As used herein, the terms “peptide,” polypeptide,” and “protein” are used interchangeably. Peptide mimetics include peptides having one or more of the following modifications:

1. peptides wherein one or more of the peptidyl —C(O)NR— linkages (bonds) have been replaced by a non-peptidyl linkage such as a —CH₂₋carbamate linkage (—CH₂OC(O)NR—), a phosphonate linkage, a —CH₂₋sulfonamide (—CH₂₋S(O)₂NR—) linkage, a urea (—NHC(O)NH—) linkage, a—CH₂-secondary amine linkage, or with an alkylated peptidyl linkage (—C(O)NR—) wherein R is C₁₋C₄ alkyl;

2. peptides wherein the N-terminus is derivatized to a—NRR₁ group, to a —NRC(O)R group, to a —NRC(O)OR group, to a —NRS(O)₂R group, to a —NHC(O)NHR group where R and R₁ are hydrogen or C₁₋C₄ alkyl with the proviso that R and R₁ are not both hydrogen;

3. peptides wherein the C terminus is derivatized to —C(O)R₂ where R₂ is selected from the group consisting of C₁₋C₄ alkoxy, and —NR₃R₄ where R₃ and R₄ are independently selected from the group consisting of hydrogen and C₁₋C₄ alkyl.

Naturally occurring amino acid residues in peptides are abbreviated as recommended by the IUPAC-IUB Biochemical Nomenclature Commission as follows: Phenylalanine is Phe or F; Leucine is Leu or L; Isoleucine is Ile or I; Methionine is Met or M; Norleucine is Nle; Valine is Val or V; Serine is Ser or S; Proline is Pro or P; Threonine is Thr or T; Alanine is Ala or A; Tyrosine is Tyr or Y; Histidine is H is or H; Glutamine is Gln or Q; Asparagine is Asn or N; Lysine is Lys or K; Aspartic Acid is Asp or D; Glutamic Acid is Glu or E; Cysteine is Cys or C; Tryptophan is Trp or W; Arginine is Arg or R; Glycine is Gly or G, and X is any amino acid. Other naturally occurring amino acids include, by way of example, 4-hydroxyproline, 5-hydroxylysine, and the like.

Synthetic or non-naturally occurring amino acids refer to amino acids which do not naturally occur in vivo but which, nevertheless, can be incorporated into the peptide structures described herein. The resulting “synthetic peptide” contains amino acids other than the 20 naturally occurring, genetically encoded amino acids at one, two, or more positions of the peptides. For instance, naphthylalanine can be substituted for tryptophan to facilitate synthesis. Other synthetic amino acids that can be substituted into peptides include L-hydroxypropyl, L-3,4-dihydrooxyphenylalanyl, alpha-amino acids such as L-alpha-hydroxylysyl and D-alpha-methylalanyl, L-alpha.-methylalanyl, beta.-amino acids, and isoquinolyl. D amino acids and non-naturally occurring synthetic amino acids can also be incorporated into the peptides. Other derivatives include replacement of the naturally occurring side chains of the 20 genetically encoded amino acids (or any L or D amino acid) with other side chains.

As used herein, the term “pharmaceutically acceptable carrier” includes any of the standard pharmaceutical carriers, such as a phosphate buffered saline solution, water, emulsions such as an oil/water or water/oil emulsion, and various types of wetting agents. The term also encompasses any of the agents approved by a regulatory agency of the US Federal government or listed in the US Pharmacopeia for use in animals, including humans.

A “polylinker” is a nucleic acid sequence that comprises a series of three or more different restriction endonuclease recognitions sequences closely spaced to one another (i.e. less than 10 nucleotides between each site).

A “polynucleotide” means a single strand or parallel and anti-parallel strands of a nucleic acid. Thus, a polynucleotide may be either a single-stranded or a double-stranded nucleic acid.

As used herein, the term “promoter/regulatory sequence” means a nucleic acid sequence which is required for expression of a gene product operably linked to the promoter/regulator sequence. In some instances, this sequence may be the core promoter sequence and in other instances, this sequence may also include an enhancer sequence and other regulatory elements which are required for expression of the gene product. The promoter/regulatory sequence may, for example, be one which expresses the gene product in a tissue specific manner.

A “constitutive promoter is a promoter which drives expression of a gene to which it is operably linked, in a constant manner in a cell. By way of example, promoters which drive expression of cellular housekeeping genes are considered to be constitutive promoters.

A “core promoter” contains essential nucleotide sequences for promoter function, including the TATA box and start of transcription. By this definition, a core promoter may or may not have detectable activity in the absence of specific sequences that enhance the activity or confer tissue specific activity.

An “inducible” promoter is a nucleotide sequence which, when operably linked with a polynucleotide which encodes or specifies a gene product, causes the gene product to be produced in a living cell substantially only when an inducer which corresponds to the promoter is present in the cell.

The term “non-native promoter” as used herein refers to any promoter that has been operably linked to a coding sequence wherein the coding sequence and the promoter are not naturally associated (i.e. a recombinant promoter/coding sequence construct).

A “tissue-specific” promoter is a nucleotide sequence which, when operably linked with a polynucleotide which encodes or specifies a gene product, causes the gene product to be produced in a living cell substantially only if the cell is a cell of the tissue type corresponding to the promoter.

As used herein, “nucleic acid,” “DNA,” and similar terms also include nucleic acid analogs, i.e. analogs having other than a phosphodiester backbone. For example, the so-called “peptide nucleic acids,” which are known in the art and have peptide bonds instead of phosphodiester bonds in the backbone, are considered within the scope of the present invention.

As used herein, the term “fragment” as applied to a nucleic acid, may ordinarily be at least about 20 nucleotides in length, typically, at least about 50 nucleotides, more typically, from about 50 to about 100 nucleotides, preferably, at least about 100 to about 200 nucleotides, even more preferably, at least about 200 nucleotides to about 300 nucleotides, yet even more preferably, at least about 300 to about 350, even more preferably, at least about 350 nucleotides to about 500 nucleotides, yet even more preferably, at least about 500 to about 600, even more preferably, at least about 600 nucleotides to about 620 nucleotides, yet even more preferably, at least about 620 to about 650, and most preferably, the nucleic acid fragment will be greater than about 650 nucleotides in length.

Unless otherwise specified, a “nucleotide sequence encoding an amino acid sequence” includes all nucleotide sequences that are degenerate versions of each other and that encode the same amino acid sequence. Nucleotide sequences that encode proteins and RNA may include introns.

As used herein, “protecting group” with respect to a terminal amino group refers to a terminal amino group of a peptide, which terminal amino group is coupled with any of various amino-terminal protecting groups traditionally employed in peptide synthesis. Such protecting groups include, for example, acyl protecting groups such as formyl, acetyl, benzoyl, trifluoroacetyl, succinyl, and methoxysuccinyl; aromatic urethane protecting groups such as benzyloxycarbonyl; and aliphatic urethane protecting groups, for example, tert-butoxycarbonyl or adamantyloxycarbonyl. See Gross and Mienhofer, eds., The Peptides, vol. 3, pp. 3-88 (Academic Press, New York, 1981) for suitable protecting groups.

As used herein, “protecting group” with respect to a terminal carboxy group refers to a terminal carboxyl group of a peptide, which terminal carboxyl group is coupled with any of various carboxyl-terminal protecting groups. Such protecting groups include, for example, tert-butyl, benzyl or other acceptable groups linked to the terminal carboxyl group through an ester or ether bond.

As used herein, the term “purified” and like terms relate to an enrichment of a molecule or compound relative to other components normally associated with the molecule or compound in a native environment. The term “purified” does not necessarily indicate that complete purity of the particular molecule has been achieved during the process. A “highly purified” compound as used herein refers to a compound that is greater than 90% pure. In particular, purified sperm cell DNA refers to DNA that does not produce significant detectable levels of non-sperm cell DNA upon PCR amplification of the purified sperm cell DNA and subsequent analysis of that amplified DNA.

“Recombinant polynucleotide” refers to a polynucleotide having sequences that are not naturally joined together. An amplified or assembled recombinant polynucleotide may be included in a suitable vector, and the vector can be used to transform a suitable host cell. A recombinant polynucleotide may serve a non-coding function (e.g., promoter, origin of replication, ribosome-binding site, etc.) as well.

A host cell that comprises a recombinant polynucleotide is referred to as a “recombinant host cell.” A gene which is expressed in a recombinant host cell wherein the gene comprises a recombinant polynucleotide, produces a “recombinant polypeptide.”

A “recombinant polypeptide” is one which is produced upon expression of a recombinant polynucleotide.

A “sample,” as used herein, refers preferably to a biological sample from a subject, including, but not limited to, normal tissue samples, diseased tissue samples, biopsies, blood, saliva, feces, semen, tears, and urine. A sample can also be any other source of material obtained from a subject which contains cells, tissues, or fluid of interest. A sample can also be obtained from cell or tissue culture.

As used herein, the term “secondary antibody” refers to an antibody that binds to the constant region of another antibody (the primary antibody).

By the term “signal sequence” is meant a polynucleotide sequence which encodes a peptide that directs the path a polypeptide takes within a cell, i.e., it directs the cellular processing of a polypeptide in a cell, including, but not limited to, eventual secretion of a polypeptide from a cell. A signal sequence is a sequence of amino acids which are typically, but not exclusively, found at the amino terminus of a polypeptide which targets the synthesis of the polypeptide to the endoplasmic reticulum. In some instances, the signal peptide is proteolytically removed from the polypeptide and is thus absent from the mature protein.

As used herein, the term “solid support” relates to a solvent insoluble substrate that is capable of forming linkages (preferably covalent bonds) with various compounds. The support can be either biological in nature, such as, without limitation, a cell or bacteriophage particle, or synthetic, such as, without limitation, an acrylamide derivative, agarose, cellulose, nylon, silica, or magnetized particles.

By the term “specifically binds,” as used herein, is meant an antibody or compound which recognizes and binds a molecule of interest (e.g., an antibody directed against a polypeptide of the invention), but does not substantially recognize or bind other molecules in a sample.

“Sperm-specific,” as used herein, refers to an antigen which is present at higher levels on sperm than other cells or is exclusively present in sperm.

The term “standard,” as used herein, refers to something used for comparison. For example, a standard can be a known standard agent or compound which is administered or added to a control sample and used for comparing results when measuring said compound in a test sample. Standard can also refer to an “internal standard,” such as an agent or compound which is added at known amounts to a sample and is useful in determining such things as purification or recovery rates when a sample is processed or subjected to purification or extraction procedures before a marker of interest is measured.

A “subject” of analysis, diagnosis, or treatment is an animal. Such animals include mammals, preferably a human.

The term “substantially pure” describes a compound, e.g., a protein or polypeptide which has been separated from components which naturally accompany it. Typically, a compound is substantially pure when at least 10%, more preferably at least 20%, more preferably at least 50%, more preferably at least 60%, more preferably at least 75%, more preferably at least 90%, and most preferably at least 99% of the total material (by volume, by wet or dry weight, or by mole percent or mole fraction) in a sample is the compound of interest. Purity can be measured by any appropriate method, e.g., in the case of polypeptides by column chromatography, gel electrophoresis, or HPLC analysis. A compound, e.g., a protein, is also substantially purified when it is essentially free of naturally associated components or when it is separated from the native contaminants which accompany it in its natural state.

A “substantially pure nucleic acid”, as used herein, refers to a nucleic acid sequence, segment, or fragment which has been purified from the sequences which flank it in a naturally occurring state, e.g., a DNA fragment which has been removed from the sequences which are normally adjacent to the fragment e.g., the sequences adjacent to the fragment in a genome in which it naturally occurs. The term also applies to nucleic acids which have been substantially purified from other components which naturally accompany the nucleic acid, e.g., RNA or DNA or proteins which naturally accompany it in the cell.

A “therapeutic” treatment is a treatment administered to a subject who exhibits signs of pathology for the purpose of diminishing or eliminating those signs.

A “therapeutically effective amount” of a compound is that amount of compound which is sufficient to provide a beneficial effect to the subject to which the compound is administered.

As used herein, the term “transgene” means an exogenous nucleic acid sequence comprising a nucleic acid which encodes a promoter/regulatory sequence operably linked to nucleic acid which encodes an amino acid sequence, which exogenous nucleic acid is encoded by a transgenic mammal.

As used herein, the term “transgenic mammal” means a mammal, the germ cells of which comprise an exogenous nucleic acid.

As used herein, a “transgenic cell” is any cell that comprises a nucleic acid sequence that has been introduced into the cell in a manner that allows expression of a gene encoded by the introduced nucleic acid sequence.

As used herein, the term “treating” includes prophylaxis of the specific disorder or condition, or alleviation of the symptoms associated with a specific disorder or condition and/or preventing or eliminating said symptoms. A “prophylactic” treatment is a treatment administered to a subject who does not exhibit signs of a disease or exhibits only early signs of the disease for the purpose of decreasing the risk of developing pathology associated with the disease. As used herein, the term “treating” includes alleviating the symptoms associated with a specific disease, disorder or condition and/or preventing or eliminating said symptoms.

The term “vaccine” as used herein is defined as material used to provoke an immune response after administration of the materials to a mammal and thus conferring immunity. Said material when inoculated into a mammal has the effect of stimulating a cellular immune response comprising a T cell response or a humoral immune response comprising a B cell response generally resulting in antibody production. The T cell response may be a cytotoxic T cell response directed against macromolecules produced by the bacteria. However, the induction of a T cell response comprising other types of T cells by the vaccine of the invention is also contemplated. A B cell response results in the production of antibody which binds to the composition. The term vaccine encompasses prophylactic as well as therapeutic vaccines. A combination vaccine is one which combines two or more vaccines. By the term “immunizing a subject against an antigen” is meant administering to the subject a composition, a protein complex, a DNA encoding a protein complex, an antibody or a DNA encoding an antibody, which elicits an immune response in the human which immune response provides protection to the human against the antigen.

A “vector” is a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer of nucleic acid into cells, such as, for example, polylysine compounds, liposomes, and the like. Examples of viral vectors include, but are not limited to, adenoviral vectors, adeno-associated virus vectors, retroviral vectors, plasmids, cosmids, lambda phage vectors, and the like.

“Expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) and viruses that incorporate the recombinant polynucleotide.

The present invention is directed to a family of kinases (the tssk kinase family) that are testis abundant and expressed predominantly if not exclusively in the male germ cells of humans and mice. More particularly the present invention is directed to tssks and the use of these proteins in modulating fertility.

The present is also directed to substrates of the tssk kinase family. In one aspect, the substrate is tsks. In one aspect, the tsks comprises the amino acid sequence SEQ ID NO:30, or biologically active fragments or homologs thereof.

The developmental expression pattern of the tssk kinases, as well as the general relevance of kinases in physiological processes has led applicants to believe that this family of testis-abundant kinases has a role in spermatogenesis. The finding that the tssk kinase family and one of the putative substrates are expressed at the same time during spermatogenesis is relevant to the potential use of these proteins as contraceptive targets. Accordingly, one aspect of the present invention is directed to the isolation of the human tssk homologs and their use in isolating agents that inhibit tssk kinase activity. Such inhibitors can then be used as contraceptive agents to inhibit fertilization.

The nucleotide sequence and amino acid sequence of human tssk 3 is provided as SEQ ID NO:3 and SEQ ID NO:6, respectively. Recently, a similar mouse protein kinase was described and identified as a testis-specific serine kinase 3 (mouse tssk 3) (Zuercher et al., 2000, Mech Dev 93, 175-7), a member of a small family of testis-specific protein kinases (Bielke et al., 1994, Gene 139, 235-9; Kueng et al., 1997, J Cell Biol 139, 1851-9). Despite this homology, both the human tssk 3 and the mouse tssk 3b cDNAs demonstrate differences with mouse tssk 3 in several amino acids.

The nucleic acid sequence and amino acid sequence of human tssk 1 is provided as SEQ ID NO:1 and SEQ ID NO:4, respectively. The nucleic acid sequence and amino acid sequence of human tssk 2 is provided as SEQ ID NO:2 and SEQ ID NO:5, respectively. A fourth member of the human tssk family is designated tssk 4. The nucleic acid and amino acid sequence of tssk 4 is provided as SEQ ID NO:19 and SEQ ID NO:20, respectively.

The nucleic acid and amino acid sequences of a human tsks are provided as SEQ ID NOs:39 and 40, respectively.

In accordance with one embodiment of the present invention a purified polypeptide is provided comprising the amino acid sequence of SEQ ID NO:4, SEQ ID NO:5, SEQ ID NO:6, SEQ ID NO:20, or SEQ ID NO:40, or an amino acid sequence that differs from SEQ ID NO:4, SEQ ID NO:5, SEQ ID NO:6, SEQ ID NO:20, or SEQ ID NO:40 by one or more conservative amino acid substitutions. In another embodiment the purified polypeptide comprises an amino acid sequence that differs from SEQ ID NO:4, SEQ ID NO:5, SEQ ID NO:6, SEQ ID NO:20, or SEQ ID NO:40 by less than 5 conservative amino acid substitutions, and in a further embodiment, by 2 or less conservative amino acid substitutions. In one embodiment the purified polypeptide comprises the amino acid sequence of SEQ ID NO:4, SEQ ID NO:5, SEQ ID NO:6, SEQ ID NO:20, or SEQ ID NO:40.

The polypeptides of the present invention may include additional amino acid sequences to assist in the stabilization and/or purification of recombinantly produced polypeptides. These additional sequences may include intra- or inter-cellular targeting peptides or various peptide tags known to those skilled in the art. In one embodiment, the purified polypeptide comprises an amino acid sequence selected from the group consisting of SEQ ID NO:4, SEQ ID NO:5, SEQ ID NO:6, SEQ ID NO:20 and SEQ ID NO:40 and a peptide tag, wherein the peptide tag is linked to the peptide sequence. Suitable expression vectors for expressing such fusion proteins and suitable peptide tags are known to those skilled in the art and commercially available. In one embodiment, the tag comprises a His tag.

In another embodiment, the present invention is directed to a purified polypeptide that comprises a fragment of a tssk or a tsks polypeptide. More particularly the tssk polypeptide fragment consists of natural or synthetic portions of a full-length polypeptide selected from the group consisting of SEQ ID NO: 4, SEQ ID NO: 5, SEQ ID NO: 6, SEQ ID NO: 9, SEQ ID NO: 20 that are capable of specific binding to their natural ligand. In one embodiment, the human tssk fragment retains its ability to bind to tsks. In one aspect, the tsks comprises a polypeptide comprising SEQ ID NO:40, or a biologically active fragment or homolog thereof.

The present invention also encompasses nucleic acid sequences that encode human tssk. In one embodiment a nucleic acid sequence is provided comprising the sequence of SEQ ID NO: 1, SEQ ID NO:2, SEQ ID NO:3, SEQ ID NO:10, SEQ ID NO: 19 or fragments thereof. In another embodiment, a purified nucleic acid sequence is provided, selected from the group consisting of SEQ ID NO:1, SEQ ID NO:2, SEQ ID NO:3 and SEQ ID NO:19.

In one embodiment, the invention provides isolated nucleic acids comprising nucleic acid sequences encoding a tsks. In one aspect, the nucleic acid sequence encodes a tsks comprising the amino acid sequence SEQ ID NO:40, or a fragment or homolog thereof. In one aspect, the nucleic acid sequence comprises SEQ ID NO:39.

The present invention is also directed to recombinant human tssk and tsks gene constructs. In one embodiment, the recombinant gene construct comprises a non-native promoter operably linked to a nucleic acid sequence selected from the group consisting of SEQ ID NO:1, SEQ ID NO:2, SEQ ID NO:3, SEQ ID NO:7, SEQ ID NO:10, SEQ ID NO:19, and SEQ ID NO:39. The non-native promoter is preferably a strong constitutive promoter that allows for expression in a predetermined host cell. These recombinant gene constructs can be introduced into host cells to produce transgenic cell lines that synthesize the tssk gene products. Host cells can be selected from a wide variety of eukaryotic and prokaryotic organisms, and two preferred host cells are E. coli and yeast cells.

In accordance with one embodiment, a nucleic acid sequence selected from the group consisting of SEQ ID NO:1, SEQ ID NO:2, SEQ ID NO:3, SEQ ID NO:19, and SEQ ID NO:39 are inserted into a eukaryotic or prokaryotic expression vector in a manner that operably links the gene sequences to the appropriate regulatory sequences, and human tssk or tsks is expressed in the appropriate eukaryotic or prokaryotic cells host cell. Suitable eukaryotic host cells and vectors are known to those skilled in the art. The baculovirus system is also suitable for producing transgenic cells and synthesizing the tssk genes of the present invention. One aspect of the present invention is directed to transgenic cell lines that contain recombinant genes that express human tssks or tsks and fragments of the human tssks or human tsks. As used herein a transgenic cell is any cell that comprises an exogenously introduced nucleic acid sequence.

In one embodiment, the introduced nucleic acid is sufficiently stable in the transgenic cell (i.e. incorporated into the cell's genome, or present in a high copy plasmid) to be passed on to progeny cells. The cells can be propagated in vitro using standard cell culture procedure, or in an alternative embodiment, the host cells are eukaryotic cells and are propagated as part of an animal, including for example, a transgenic animal. In one embodiment the transgenic cell is a human cell and comprises a nucleic acid sequence selected from the group consisting of SEQ ID NO: 1, SEQ ID NO:2, SEQ ID NO:3, SEQ ID NO:7, SEQ ID NO:19, and SEQ ID NO:39. In one embodiment, the transgenic cell comprises a recombinant nucleic acid sequence, wherein the recombinant nucleic acid sequence or a fragment thereof operably linked to a non-native promoter. The present invention also includes non-human transgenic organisms wherein one or more of the cells of the transgenic organism comprise a recombinant gene that expresses a human tssk and/or tsks product.

The present invention also encompasses a method for producing human tssks and tsks. The method comprises the steps of introducing a nucleic acid sequence, comprising a promoter operably linked to a sequence that encodes a human tssk, into a host cell, and culturing the host cell under conditions that allow for expression of the introduced human tssk gene. In one embodiment the promoter is a conditional or inducible promoter, alternatively the promoter may be a tissue specific or temporal restricted promoter (i.e. operably linked genes are only expressed in a specific tissue or at a specific time). The synthesized tssks and tsks can be purified using standard techniques and used in high throughput screens to identify inhibitors of tssk and/or tsks activity. Alternatively, in one embodiment the recombinantly produced polypeptides, or fragments thereof are used to generate antibodies against the polypeptides. The recombinantly produced proteins can also be used to obtain crystal structures. Such structures would allow for crystallography analysis that would lead to the design of specific drugs to inhibit tssk or tsks.

Preferably, the nucleic acid sequences encoding the sperm-specific kinase or tsks are inserted into a suitable expression vector in a manner that operably links the gene sequences to the appropriate regulatory sequences for expression in the preselected host cell. Suitable host cells, vectors and methods of introducing the DNA constructs into cells are known to those skilled in the art. In particular, nucleic acid sequences encoding the sperm-specific kinase may be added to a cell or cells in vitro or in vivo using delivery mechanisms such as liposomes, viral based vectors, or microinjection.

The present invention provides antisense oligonucleotides useful for inhibiting expression tssk and tsks proteins.

In accordance with one embodiment, a composition is provided comprising a purified tssk or tsks peptide of the invention, or an antigenic fragment thereof. The compositions can be combined with a pharmaceutically acceptable carrier or adjuvants and administered to a mammalian species to induce an immune response.

Another embodiment of the present invention is directed to antibodies specific for the individual tssk or tsks isotypes. In one embodiment, the antibody is a monoclonal antibody. The antibodies or antibody fragments of the present invention can be combined with a carrier or diluent to form a composition. In one embodiment, the carrier is a pharmaceutically acceptable carrier. Such carriers and diluents include sterile liquids such as water and oils, with or without the addition of a surfactant and other pharmaceutically and physiologically acceptable carrier, including adjuvants, excipients or stabilizers. Illustrative oils are those of petroleum, animal, vegetable, or synthetic origin, for example, peanut oil, soybean oil, or mineral oil. In general, water, saline, aqueous dextrose, and related sugar solution, and glycols such as, propylene glycol or polyethylene glycol, are preferred liquid carriers, particularly for injectable solutions.

Antibodies to human tssks and tsks may be generated using methods that are well known in the art. In accordance with one embodiment, an antibody is provided that specifically binds to a polypeptide selected from the group consisting of SEQ ID NO:4, SEQ ID NO:5, SEQ ID NO:6, SEQ ID NO:8, SEQ ID NO:9, SEQ ID NO:20, and SEQ ID No:40. The antibodies may be used with or without modification, and may be labeled by joining them, either covalently or non-covalently, with a reporter molecule. In addition, the antibodies can be formulated with standard carriers and optionally labeled to prepare therapeutic or diagnostic compositions.

For the production of antibodies, various host animals, including but not limited to rabbits, mice, rats, etc can be immunized by injection with a polypeptide of the invention or peptide fragment thereof. Various adjuvants may be used to increase the immunological response, depending on the host species, and including but not limited to Freund's (complete and incomplete), mineral gels such as aluminum hydroxide, surface active substances such as lysolecithin, pluronic polyols, polyanions, peptides, oil emulsions, keyhole limpet hemocyanins, dinitrophenol, and potentially useful human adjuvants such as BCG (bacille Calmette-Guerin) and corynebacterium parvum.

For preparation of monoclonal antibodies, any technique which provides for the production of antibody molecules by continuous cell lines in culture may be used. For example, the hybridoma technique originally developed by Kohler and Milstein (1975, Nature 256:495-497), as well as the trioma technique, the human B-cell hybridoma technique (Kozbor et al., 1983, Immunology Today 4:72), and the EBV-hybridoma technique to produce human monoclonal antibodies (Cole et al., 1985, in Monoclonal Antibodies and Cancer Therapy, Alan R. Liss, Inc., pp. 77-96). In an additional embodiment of the invention, monoclonal antibodies can be produced in germ-free animals utilizing recent technology (PCT/US90/02545). According to the invention, human antibodies may be used and can be obtained by using human hybridomas (Cote et al., 1983, Proc. Natl. Acad. Sci. U.S.A. 80:2026-2030) or by transforming human B cells with EBV virus in vitro (Cole et al., 1985, in Monoclonal Antibodies and Cancer Therapy, Alan R. Liss, pp. 77-96). In fact, according to the invention, techniques developed for the production of “chimeric antibodies” (Morrison et al., 1984, Proc. Natl. Acad. Sci. U.S.A. 81:6851-6855; Neuberger et al., 1984, Nature 312:604-608; Takeda et al., 1985, Nature 314:452-454) by splicing the genes from a mouse antibody molecule specific for epitopes of the peptide of interest together with genes from a human antibody molecule of appropriate biological activity can be used; such antibodies are within the scope of this invention.

According to the invention, techniques described for the production of single chain antibodies (U.S. Pat. No. 4,946,778) can be adapted to produce egg surface protein-specific single chain antibodies. An additional embodiment of the invention utilizes the techniques described for the construction of Fab expression libraries (Huse et al., 1989, Science 246:1275-1281) to allow rapid and easy identification of monoclonal Fab fragments with the desired specificity for tssk epitopes.

Antibody fragments which contain the idiotype of the molecule can be generated by known techniques. For example, such fragments include but are not limited to: the F(ab′)₂ fragment which can be produced by pepsin digestion of the antibody molecule; the Fab′ fragments which can be generated by reducing the disulfide bridges of the F(ab′)₂ fragment, the Fab fragments which can be generated by treating the antibody molecule with papain and a reducing agent, and Fv fragments.

In the production of antibodies, screening for the desired antibody can be accomplished by techniques known in the art, e.g. ELISA (enzyme-linked immunosorbent assay). The foregoing antibodies can be used in methods known in the art relating to the localization and activity of the tssk or tsks proteins of the invention, e.g., for imaging these proteins, measuring levels thereof in appropriate physiological samples, in diagnostic methods, etc. Antibodies generated in accordance with the present invention may include, but are not limited to, polyclonal, monoclonal, chimeric (i.e. “humanized” antibodies), single chain (recombinant), Fab fragments, and fragments produced by a Fab expression library.

The present invention also provides a method for detecting the presence of human tssk and tsks. The method comprises the steps of contacting a sample with a labeled antibody that specifically binds to human tssk or tsks, removing unbound and non-specific bond material and detecting the presence of the labeled antibody. In one embodiment the labeled compound comprises an antibody that is labeled directly or indirectly (i.e. via a labeled secondary antibody). In particular, the tssk antibodies of the present invention can be used to confirm the expression of tssk and/or tsks as well as its cellular location, or in assays to monitor individuals receiving a tssk and/or tsks inhibitory composition as a means of contraception.

Tssks and tsks are highly testis abundant, if not exclusively produced in the testis. This makes the tssk kinases and tsks optimal targets for the identification and development of drugs that modulate its activity to study the role of tssks in spermiogenesis. Furthermore, inhibitors of tssk and/or tsks synthesis, levels, and activity are anticipated to have utility as contraceptive agents. In accordance with one aspect of the present invention, the tssk kinase family is used as a target for the identification and development of novel drugs or drugs which inhibit tssk synthesis, levels, or activity. In one aspect, the target is a tsks. Progress in the field of small molecule library generation, using combinatorial chemistry methods coupled to high-throughput screening, has accelerated the search for ideal cell-permeable inhibitors. In addition, structural-based design using crystallographic methods has improved the ability to characterize in detail ligand-protein interaction sites that can be exploited for ligand design.

In one embodiment, the present invention provides methods of screening for agents, small molecules, or proteins that interact with polypeptides comprising the sequence of tssk 1, tssk 2, tssk 3, tssk 4, or a tsks, or bioactive fragments thereof. As used herein, the term “biologically active fragments” or “bioactive fragment” of tssk 1, tssk 2, tssk 3 and tssk 4, and tsks, encompasses natural or synthetic portions of the native peptides that are capable of specific binding to at least one of the natural ligands of the respective native tssk 1, tssk 2, tssk 3 and tssk 4 polypeptide, including tsks. The invention encompasses both in vivo and in vitro assays to screen small molecules, compounds, recombinant proteins, peptides, nucleic acids, antibodies etc. which bind to or modulate the activity of tssk 1, tssk 2, tssk 3 and tssk 4 and are thus useful as therapeutic or diagnostic markers for fertility.

In one embodiment of the present invention tssk polypeptides, selected from the group consisting of SEQ ID NO:4, SEQ ID NO:5, SEQ ID NO:6 and SEQ ID NO:20 are used to isolate ligands that bind to a tssk under physiological conditions. The screening method comprises the steps of contacting a tssk or tsks polypeptide with a mixture of compounds under physiological conditions, removing unbound and non-specifically bound material, and isolating the compounds that remain bound to the tssk polypeptide. Typically, the tssk or tsks polypeptide will be bound to a solid support, using standard techniques, to allow for rapid screening of compounds. The solid support can be selected from any surface that has been used to immobilize biological compounds and includes but is not limited to polystyrene, agarose, silica or nitrocellulose. In one embodiment the solid surface comprises functionalized silica or agarose beads. Screening for such compounds can be accomplished using libraries of pharmaceutical agents and standard techniques known to the skilled practitioner.

Ligands that bind to the tssk and tsks polypeptides can then be further analyzed for agonist and antagonist activity through the use of an in vitro kinase assay such as that described herein or other assays known to those of skill in the art. Inhibitors of tssk kinase and tsks synthesis, levels, and activity have potential use as agents that prevent maturation/capacitation of sperm. In accordance with one embodiment, inhibitors of tssks and tsks are isolated as potential contraceptive agents. Such inhibitors can be formulated as pharmaceutical compositions and administered to a subject to block spermatogenesis and provide a means for contraception.

In accordance with one embodiment, specific inhibitors of human tssk kinase activity are identified through the use of an in vitro kinase assay that is capable to detecting phosphorylation events. In one embodiment the method of identifying inhibitors of tssk kinase activity comprises combining a labeled source of phosphate with one or more of the human tssk polypeptides in the presence of one or more potential inhibitory compounds. The reactions are conducted under standard conditions that in the absence of the potential inhibitory compound are suitable for initiating the phosphorylation of the tssk substrate (i.e., tsks) or the tssk enzyme itself.

Decreases in tssk activity can be detected by comparing the amount of phosphorylated tssk substrate in the assay relative to a standard curve plotting kinase activity vs. time. Alternatively an inhibitory decrease in kinase activity can also be detected by conducting a second kinase reaction wherein an in vitro kinase assay composition, comprising a labeled source of phosphate, a tssk substrate and a tssk kinase, is incubated under conditions permissive for kinase activity using identical conditions as that used for the test assay (the assay conducted in the presence to potential inhibitory compounds). The amount of phosphorylated tssk substrate produced by the test assay is then compared to the amount of phosphorylated tssk substrate produced by the second kinase assay (conducted in the absence of the candidate inhibitor) to detect a tssk inhibitory effect of the candidate compound.

In one embodiment, specific inhibitors of tssk and tsks synthesis and levels are identified using assays known to those of skill in the art.

Once compounds have been identified that inhibit tssk and/or tsks synthesis, levels, and activity, further testing will need to be conducted to isolate those compounds. In accordance with one embodiment the method of identifying tssk and tsks inhibitors further comprises the step of conducting a second reaction wherein the candidate compound is contacted with a control composition wherein the control composition comprises a the candidate compound, a labeled source of phosphate, a kinase substrate and a non-tssk kinase, and determining if the candidate compound decreases the activity of the non-tssk kinase.

One embodiment of the present invention is directed to decreasing the fertility of a male mammal, said method comprising the steps of inhibiting the synthesis, levels, and activity of the tssk kinase family (including tssk 1, tssk 2, tssk 3 and tssk 4) and/or tsks. In one embodiment, the fertility of a male mammal is decreased by the administration of a pharmaceutical composition that comprises an agent that specifically interferes with tssk activity.

In one embodiment, the fertility inhibiting composition comprises a chemical entity that specifically inhibits the enzymatic activity of one or more of the tssk kinases.

In another embodiment the inhibitory composition may comprise an antibody against one or more of the tssk kinases or the composition may comprise an antisense or interference RNA that prevents or disrupts the expression of the tssk kinases in an animal. Interference RNA in mammalian systems requires the presence of short interfering RNA (siRNA), which consists of 19-22 nt double-stranded RNA molecules, or shRNA, which consists of 19-29 nt palindromic sequences connected by loop sequences. Down regulation of gene expression is achieved in a sequence-specific manner by pairing between homologous siRNA and target RNA. A system for the stable expression of siRNA or shRNA was utilized to generate transgenic animals (Hasuwa et al. FEBS Lett 532, 227-30 (2002), Rubinson et al. Nat Genet. 33, 401-6 (2003) and Carmell et al. Nat Struct Biol 10, 91-2 (2003)) and can be used in accordance with the present invention to produce animals whose fertility can be regulated. A conditional RNAi-based transgenic system would provide the additional benefit of being able to control the level of gene expression at any given stage during the life of the animal.

### EXAMPLE 1

### Validation of TSSKs as Male Contraceptive Targets

Methods—

The methods used herein are known to those of skill in the art and are described in, inter alia, U.S. Pat. Nos. 6,946,275, 6,924,121, 6,355,480, and 6,258,364, U.S. Patent Publication Nos. 20050032146, 20040161825, 20040161824, 20040030112, and 20030211563, Hao et al., Molecular Human Reproduction, 10:6:433-444, 2004, Scorilas et al., Biochem. Biophys. Res. Commun., 285:2:400-408, 2001, and Spiridonov et al., 2005, Molecular and Cellular Biology, 25:10:4250-4261, the methods of which are incorporated herein in their entirety.

Results—

A family of testis specific serine/threonine kinases, TSSK 1-4, is being investigated with the goal of validation of these genes/proteins as male contraceptive targets. TSSK1, TSSK2, TSSK3, and TSSK4 constitute four members of a unique kinase subfamily that, to date, have been cloned in both the human and mouse. Each member contains 12 kinase subdomains, and each is predicted to be an active kinase. TSSK1 and TSSK2 have higher homology and longer carboxyl termini than TSSK3 and TSSK4. Human TSSK1, TSS1, TSSK3 and TSSK4 map to chromosomes 5, 22, 1 and 19, respectively, with an additional pseudogene located 3 kb upstream of TSSK2. Mouse TSSK1 and TSSK2 are closely linked on chromosome 16 with only a 3 kb intergenic region, offering the possibility of creating a knockout mouse of both TSSK1 and TSSK with a single targeted construct. Mouse TSSK3 and TSSK4 map to chromosomes 5 and 19, respectively.

Expression profiling showed that all four kinases are testis abundant, if not strictly testis specific, an indication that tissue specific contraceptive targeting may be possible. Real-time PCR demonstrated that the transcripts of TSSK1 and TSSK2 are approximately 10 times more abundant than TSSK3 and TSSK4 in human testis.

In situ hybridization further suggested that TSSK2 and TSSK4 kinases may be post-meiotic in their expression patterns, an observation that supports a possible application in reversible contraceptive intervention by preserving spermatogonia and spermatocytes. Polyclonal antibodies demonstrated TSSK and TSSK4 proteins in the testis and sperm by western analysis, and localized them to the equatorial segment in sperm by immunofluorescence.

A plasmid targeted for a double knock out TSSK1 and TSSK2 in the mouse has been constructed. ES cell transfection and mutant mouse generation are being performed. Future work will study the effect of targeted disruption of the murine TSSK1 and TSSK2 genes on spermatogenesis and sperm function.

TSSK1-4 constitute a testis specific serine/threonine kinase subfamily (see Table 1.

TABLE 1
The mammalian TSSKs in the genome

| Gene | Gene | | | | |
|---|---|---|---|---|---|
| name | name | Chromosome | Chromosome | Intron? | Intron? |
| mouse | human | Mouse | Human | Mouse | Human |
| STK22A | TSSK1 | 16 10.4 cM | 5q22.2 | no | no |
| — | TSSK1- | — | 22q11.21 | — | no |
| | pseudo | | | | |
| STK22B | TSSK2 | 16 10.4 cM | 22q11.21 | no | no |
| STK22C | TSSK3 | 4cytoband | 1p35.1 | yes | yes |
| | | D2.3 | | | |
| SSTK | TSSK4 | 8cytoband C1 | 19p13.11 | no | yes |

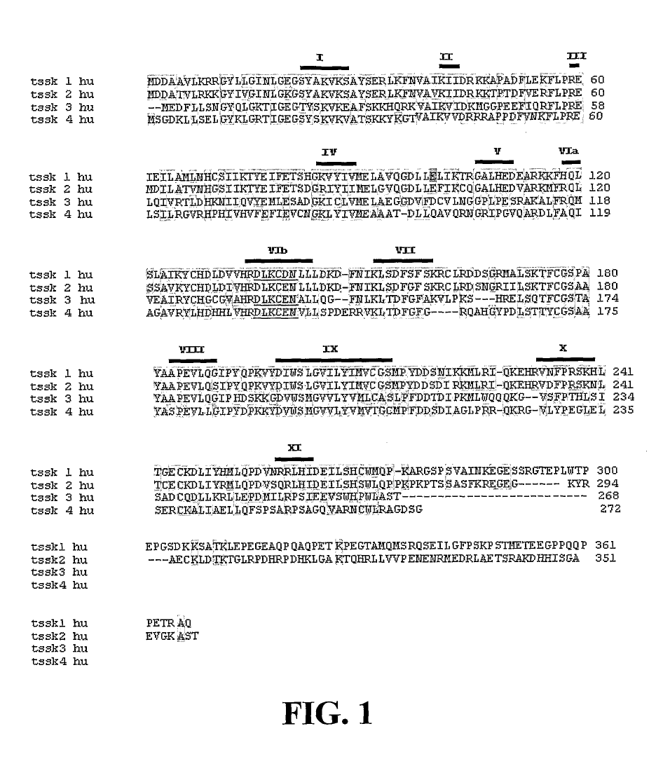

FIG. 1 further provides a sequence alignment comparison of TSSK1, TSSK2, TSSK3, and TSSK4 amino acid sequences. The highest homology is found between TSSK1 and TSSK (83% in the kinase domain and 72% across the entire ORF). TSSK3 is 47.5% and 49% identical to TSSK1 and TSSK respectively. TSSK4 is 49% identical to TSSK3. The highly conserved signature sequence that fits the consensus “DLKXXN” for serine/threonine kinases is underlined.

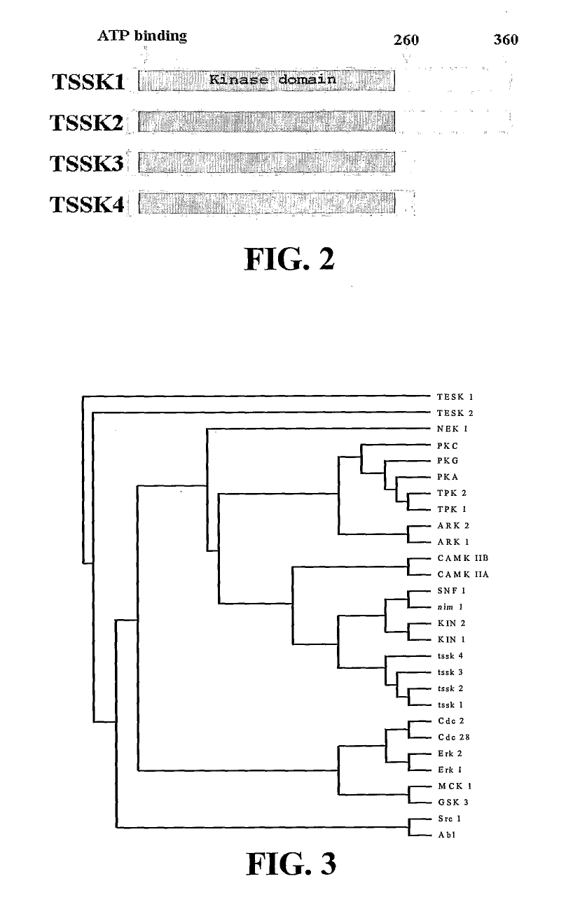

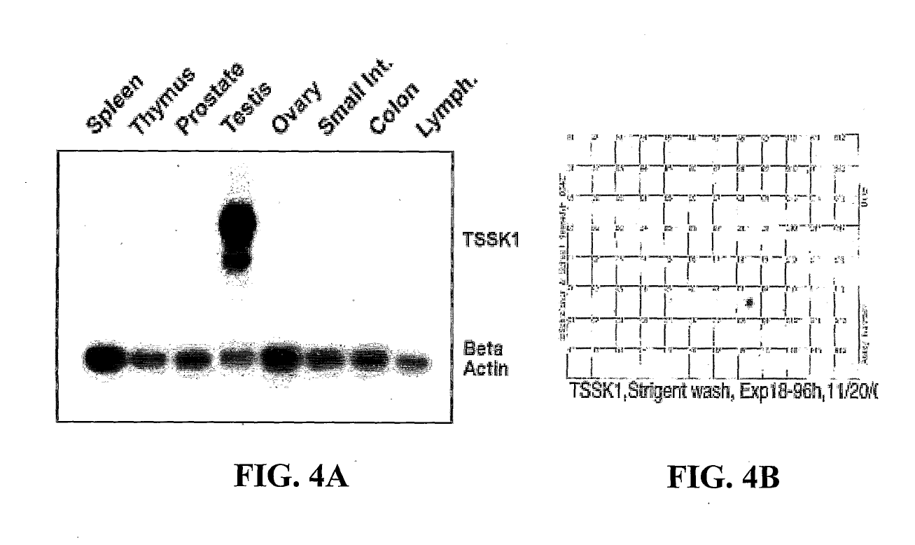

The domain structure of the mammalian TSSK subfamily is illustrated in FIG. 2. TSSK1-4 are predicted to be active kinases, the kinase domains compose the major part of the TSSKs. TSSK1 and TSSK2 are longer at their carboxyl termini than TSSK 3 and TSSK4. The Phylogenetic relationship of TSSKs with other kinases, and the relationship between the TSSK family members are provided in FIG. 3. TSSK 1 and TSSK form a subgroup within the TSSK family.

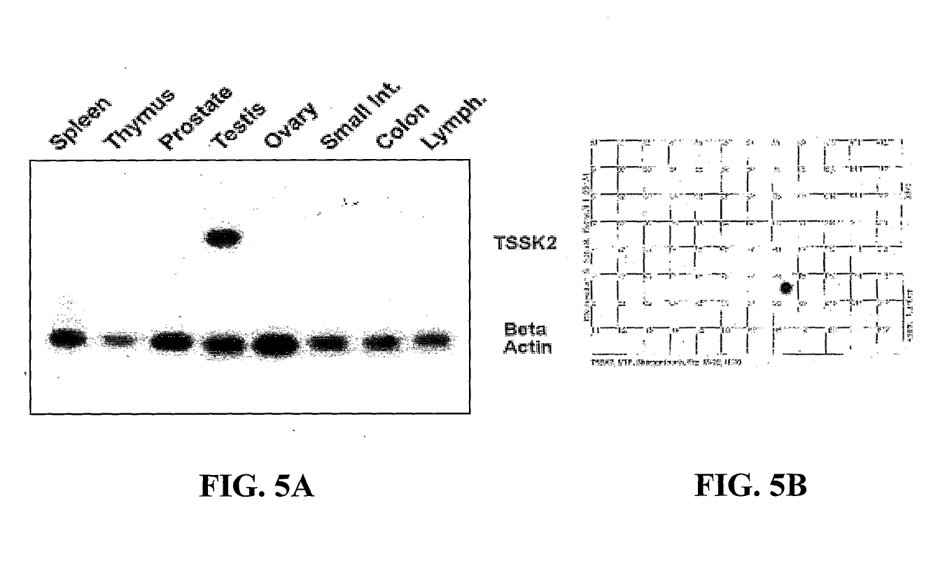

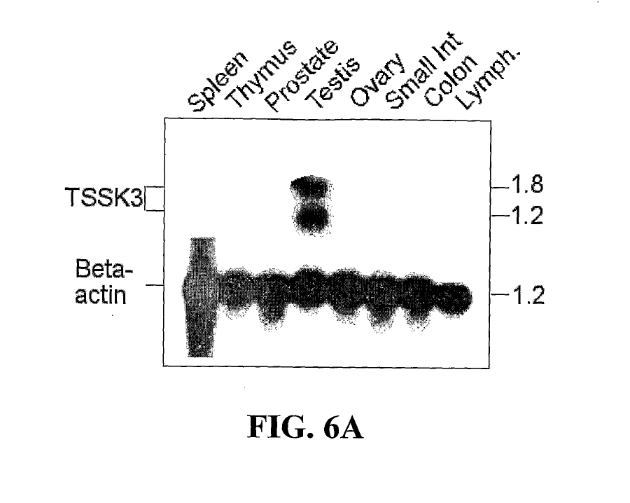

Expression results in 72 tissues indicate that TSSK1 transcripts were only located in testis (see FIG. 4), as were TSSK 2 transcripts (FIG. 5).

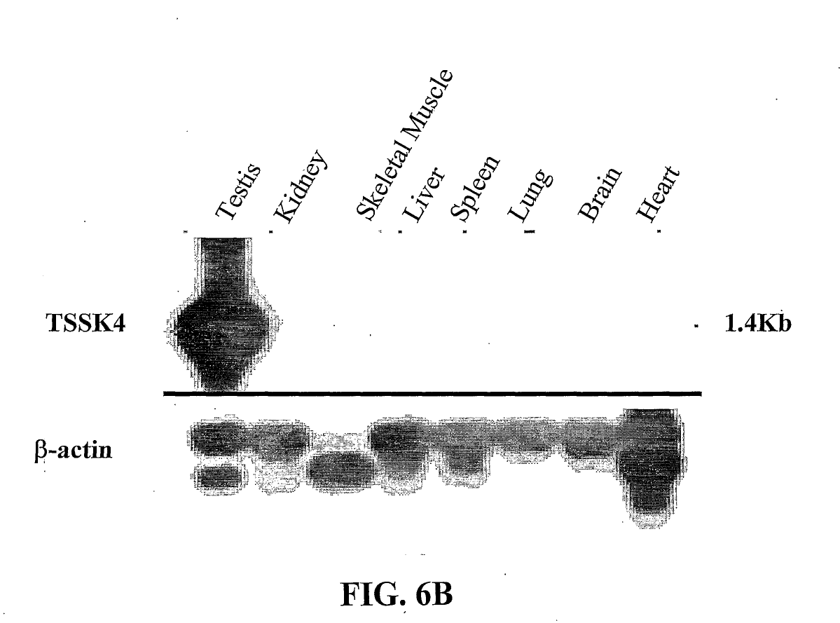

Expression results in eight tissues indicate that human TSSK3 (FIG. 6A) and mouse TSSK4 (FIG. 6B) were only expressed in testis. It can be seen in FIG. 6A that TSSK3 mRNA may have multiple splice sites.

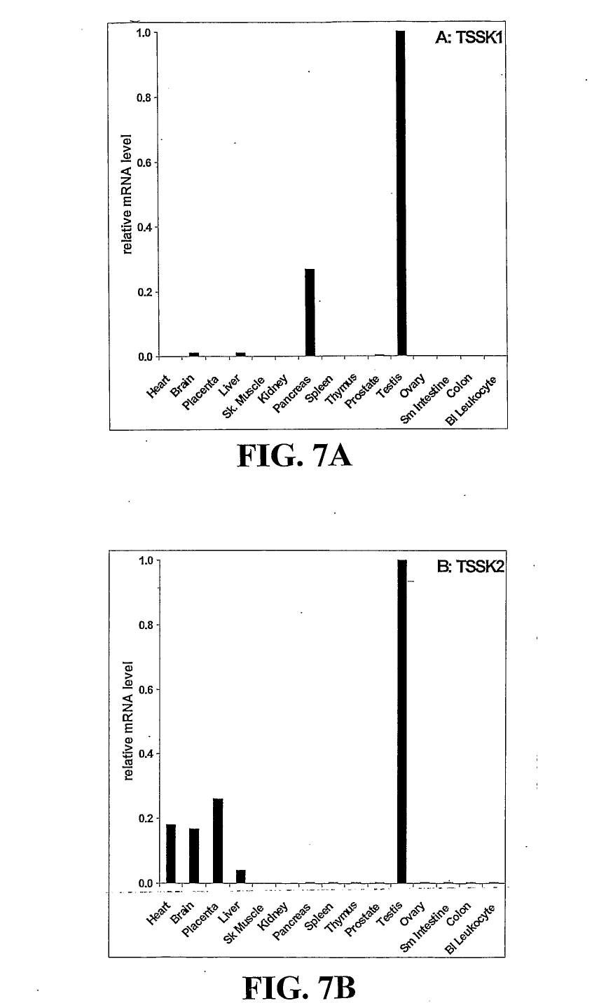

Real time PCR analysis (FIGS. 7A, 7B, 7C, and 7D) studied TSSK1 (7A), TSSK (7B), TSSK3 (7C), and TSSK4 (7D) expression in fifteen human tissues. TSSK 3 and 4 transcripts were detected only in testis, while TSSK 1 was also detectable in pancreas, and TSSK2 was also detectable in heart, brain, placenta, and liver.

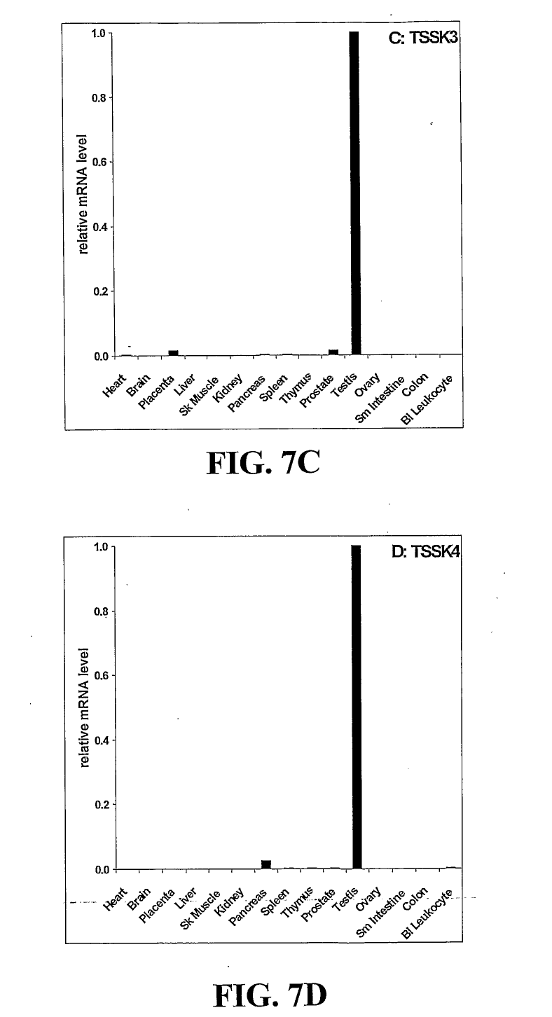

Real-time PCR quantitation of human TSSK1-4 relative expression levels in testis demonstrated TSSK1 and 2 transcripts are approximately 10 times more abundant than TSSK 3 and 4 in human testis (see FIG. 8).

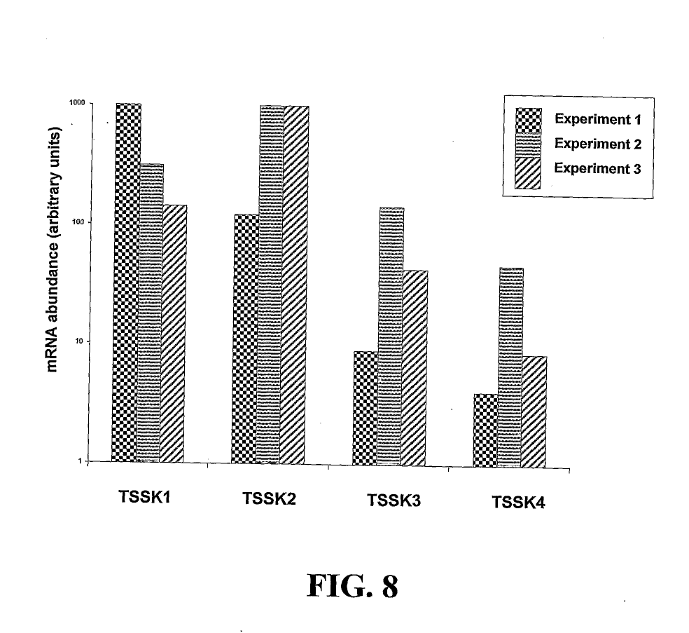

Experiments to construct knockout mice utilize the strategy of FIG. 9.

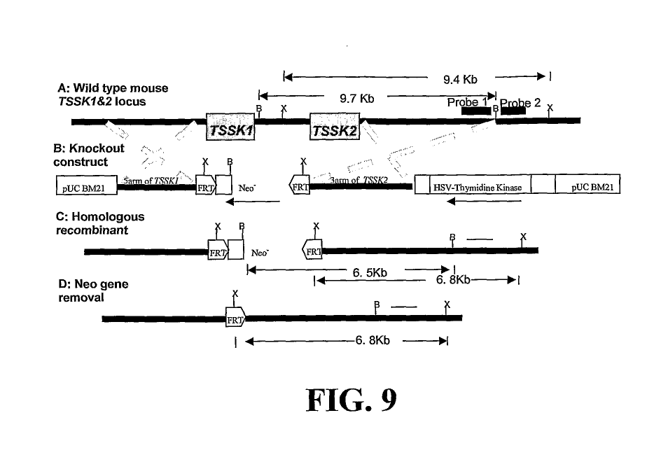

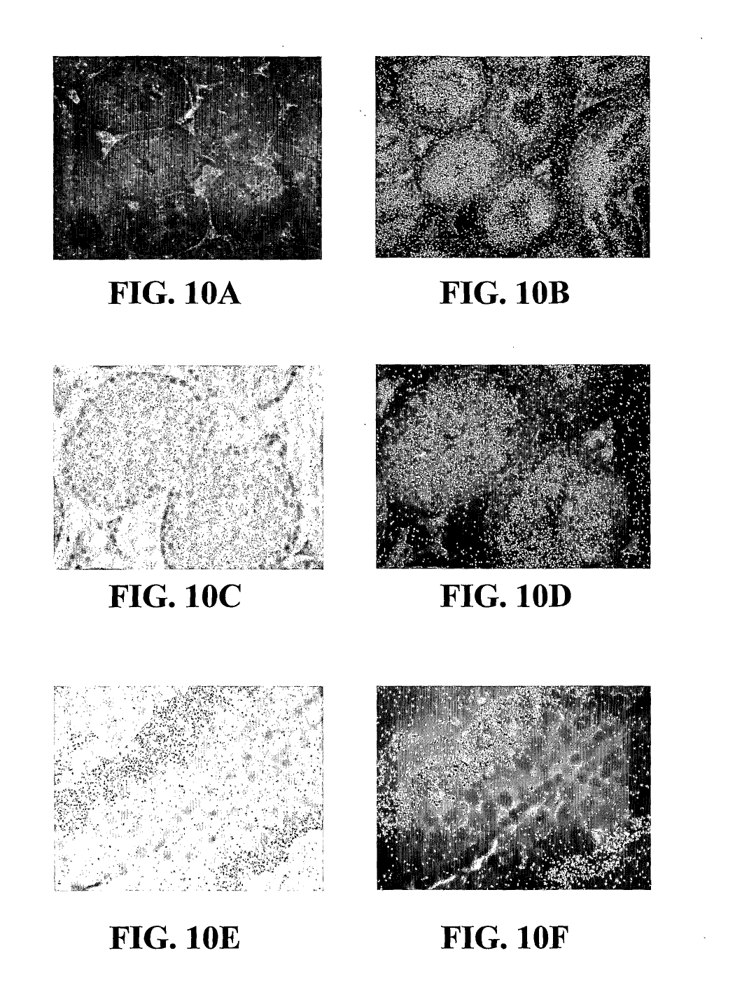

The results of experiments determining expression in situ are illustrated in FIG. 10, which represents photomicrographic images of in situ analyses of TSSK mRNA expression in adult mouse testis, indicating post-meiotic expression and sperm equatorial segment localization of TSSK2. In situ hybridization of TSSK2 transcripts was carried out using radiolabeled mouse TSSK2 cRNA. The low-magnification (×100) views of seminiferous tubules hybridized with sense TSSK2 (10A) or antisense TSSK2 (10B) are shown in dark field, the higher magnification (×200, ×400) views of seminiferous tubules hybridized with antisense TSSK2 are also shown in both bright-field (10C and 10D) and dark field (10E and 10F). TSSK2 transcripts are expressed mainly in the post-meiotic spermatids, while the labeling on the primary spermatocytes is not much greater than the background. Expression of TSSK4 is demonstrated in FIG. 11, demonstrating TSSK4 mRNA expression (in adult mouse testis) post-meiotic expression and sperm equatorial segment localization of TSSK4. In situ hybridization of TSSK4 transcripts was carried out using radiolabeled mouse TSSK4 cRNA. The low-magnification (×100) views of seminiferous tubules hybridized with sense TSSK4 (11A) or antisense TSSK4 (11B) are shown in dark field, the higher magnification (×200, ×400) views of seminiferous tubules hybridized with antisense TSSK4 are shown in both bright-field (11C and 11D), and dark field (11E and 11F). TSSK4 transcripts were expressed mainly in the post-meiotic spermatids.

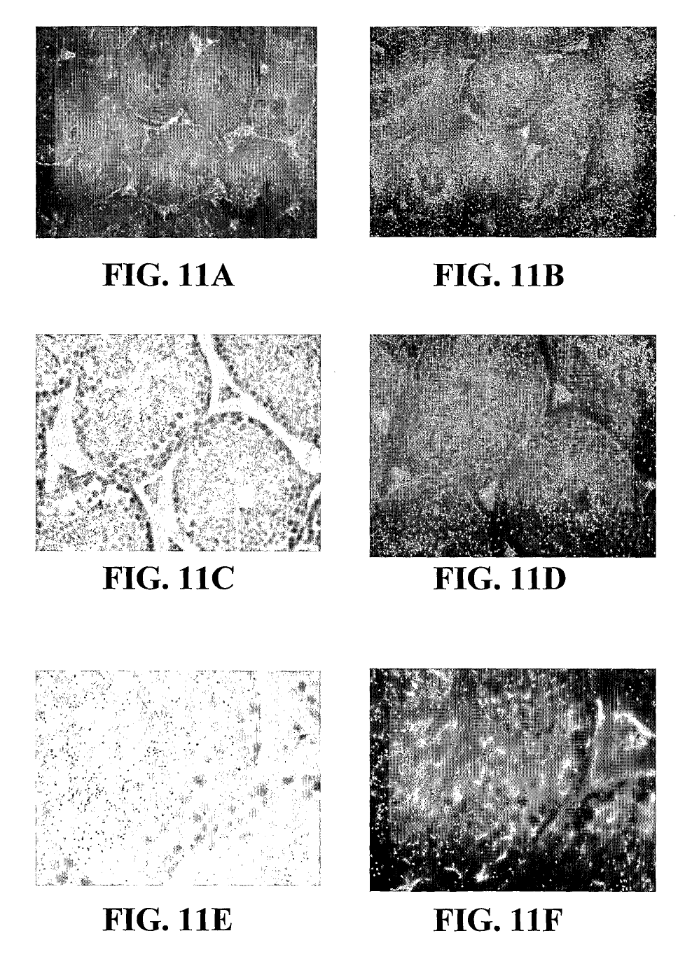

The results of a western blot analysis of TSSK2 expression in human testis and sperm are provided in FIG. 12.

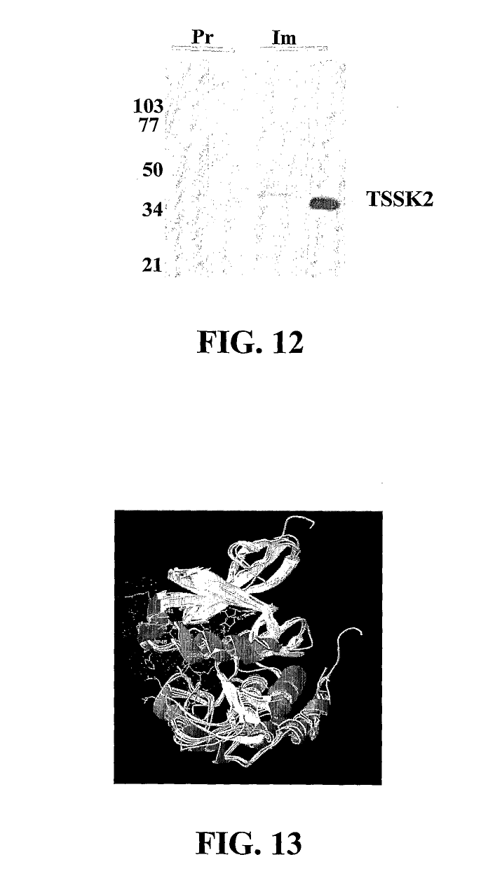

An analysis was performed to model TSSK4. FIG. 13 schematically represents the 3D structure of mouse TSSK4 modeled by computer. A peptide (RRAPPDFVNKFLPRE) identical in mouse and human TSSK4 proteins was used to generate antibody against TSSK4. Mouse TSSK4 structure was modeled to show that this peptide is on the surface of the molecule after folding.

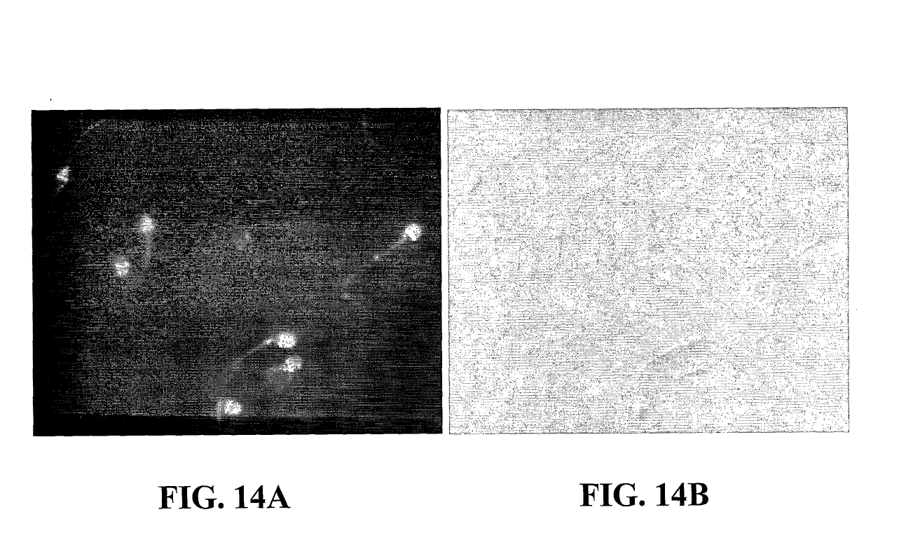

Immunofluorescent localization of TSSK2 in ejaculated sperm was performed (FIG. 14). TSSK2 was localized weakly to the acrosomal cap and more intensely to the equatorial segment. Its localization to the equatorial segment of the sperm suggests a role for TSSK2 during fertilization.

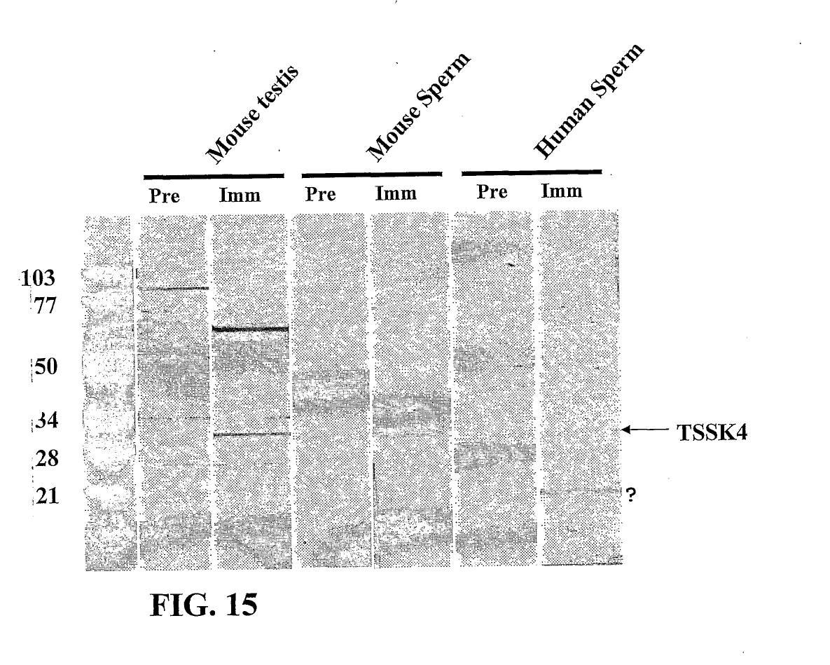

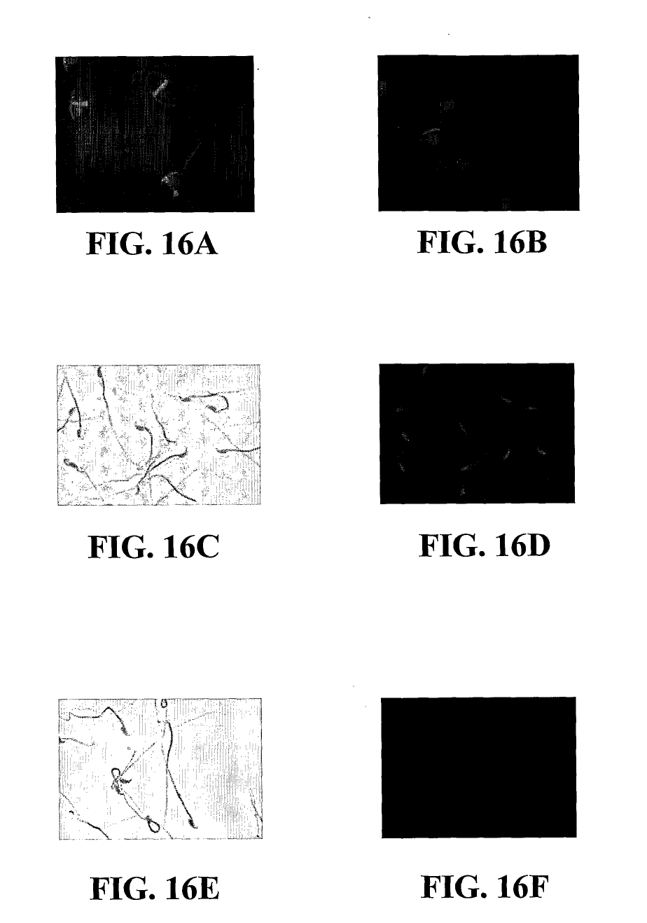

A Western blot analysis of TSSK4 expression in mouse testis, mouse sperm, and human sperm was performed (FIG. 15). A single band with the predicted molecular size of TSSK4 was detected in mouse sperm protein extracts using rabbit TSSK4 anti-serum, while a lower molecular weight TSSK4 protein, a likely proteolytic product, was detected in human sperm protein extracts. The higher molecular weight band in the protein extracts from mouse testis may be a TSSK4 dimer.

Immunofluorescent localization of TSSK4 in mouse and human sperm was performed. TSSK4 was localized to the equatorial segment in both mouse and human sperm. Its localization to the equatorial segment of the sperm might indicate a role for TSSK4 during fertilization.

### TSSK Summary

TSSK1, 2, 3 and 4 constitute a unique subfamily of serine/threonine kinases.

TSSK 3 and TSSK4 are testis specific genes, implying that tissue specific contraceptive targeting may be possible.

The TSSK1 messages in pancreas and TSSK2 messages in heart, brain, and placenta require confirmation at protein levels.

TSSK2 and 4 kinases are post-meiotic in their expression patterns, which underscores a possible application in reversible contraceptive intervention by preserving spermatogonia and spermatocytes.

TSSK2 and 4 are found in the testis and sperm by western analysis, and were localized to the equatorial segment in sperm by immunofluorescence. Their localization to the equatorial segment of the sperm might indicate a role during fertilization.

A plasmid targeted for a double knock out TSSK1 and 2 in the mouse has been constructed. ES cell transfection and mutant mouse generation are in progress.

### EXAMPLE 2

### Validation of TSKS as a Male Contraceptive Target

TSKS, the substrate of TSSK1 and TSSK, is being investigated with the goal of identifying small molecule inhibitors that may target the interaction between these kinases and their substrate. Human TSKS has been cloned and mapped to chromosome 19. Northern and RNA dot blot analyses indicated that TSKS transcripts were exclusively expressed in the testis, an indication that tissue specific contraceptive targeting may be possible.

Polyclonal antibodies against both human and mouse TSKS were generated. Western analysis demonstrated that TSKS is restricted to testis, and is absent in mature sperm. Immunofluorescent localization of TSKS within disassociated human testicular cells revealed TSKS within round, elongating and elongated spermatids while being virtually absent in mature testicular spermatids TSKS proteins are first detected in the round spermatids, and the greatest fluorescent peak in the elongating spermatids. TSKS is located at the base of condensing head of the spermatid as two fluorescent spots, corresponding to the centriole of the sperm neck. In situ hybridization further suggested that TSKS messages are post-meiotic in their expression pattern. This result underscores the possibility of a reversible contraceptive intervention targeted to TSKS or its interaction with TSSK 1 or 2 while preserving spermatogonia and spermatocytes. Binding interactions between human TSKS and TSSK2 were confirmed by the yeast two hybrid system, and in-vitro co-immunoprecipitation. The N-terminus of TSKS was shown to be essential for interaction with TSSK2. Human recombinant TSKS in E. coli and TSSK2 in yeast have been expressed, paving the way for phospho-peptide mapping.

Mouse TSKS immunoprecipitated from testis was shown to be phosphorylated in an in-vitro kinase assay, which provides a high throughput screen for inhibitors of TSKS phosphorylation. TSSK is autophosphorylated in the in-vitro kinase assay. In sum, the TSKS/TSSK1 and 2 substrate/kinases offer the possibility of post-meiotic testicular targeting.

The human TSKS nucleic acid sequence (SEQ ID NO:39) and amino acid sequence (SEQ ID NO:40) are provided below. The mouse nucleic acid sequence (SEQ ID NO:42) and amino acid sequence (SEQ ID NO:41) are provided below. The nucleic acid and amino acid sequences for human and mouse TSKS are:

| Human TSKS, NCBI accession number NM_021733 | |
|---|---|
| (SEQ ID NO:39) | |
| 1 | acccccacac catggcgagc gtggtggtga agacgatctg gcagtccaaa gagatccatg |
| 61 | aggccgggga cacccccacg ggggtggaga gctgctccca gctagtccca gaggctcccc |
| 121 | ggagggtgac cagccgggcc aaggggatcc cgaagaaaaa gaaggccgtg tcgttccacg |
| 181 | gggtggagcc ccagatgtcc catcagccca tgcactggtg cctgaacctc aaacggtcct |
| 241 | cggcctgcac caacgtgtca ctgctcaacc tggccgccat ggagcccact gactccacgg |
| 301 | ggacagactc cacagtggaa gacctcagcg gccaactcac actggctggg ccccctgcct |
| 361 | cccctaccct accctgggat ccggatgacg cagacatcac ggaaatcctg agtggggtca |
| 421 | acagtggatt ggtccgcgcc aaagactcca tcaccagctt gaaggaaaag accaaccggg |
| 481 | ttaaccagca cgtgcagtct ctgcagagcg agtgttctgt gctgagcgag aatctggaga |
| 541 | gaaggcggca agaggcagaa gagttggagg ggtactgcat tcaactcaag gagaactgct |
| 601 | ggaaggtgac ccggtctgtg gaagatgctg aaatcaaaac caacgtcttg aagcagaatt |
| 661 | ctgccctgct ggaggagaag ctgcgctacc tccagcagca gctgcaggat gagacgccgc |
| 721 | gacggcagga ggccgagctg caggagccgg aggagaagca ggagccggag gagaagcagg |
| 781 | agccggagga gaagcagaag ccggaggctg gcctctcctg gaacagcctg ggccccgccg |
| 841 | ccacgtccca gggctgcccc ggcccgccag ggagtcccga caaaccctcg cggccacacg |
| 901 | gcctggtccc cgcaggctgg ggaatggggc ctcgggctgg cgagggcccc tacgtgagcg |
| 961 | agcaggaatt gcagaagctg ttcaccggca tcgaagagct gaggagagag gtgtcctcac |
| 1021 | tgaccgcccg gtggcatcag gaggaggggg cggtgcagga agccctgcgg ctgctcgggg |
| 1081 | gcctgggcgg cagggtcgac ggcttcctag gccagtggga gcgggcacag cgcgaacagg |
| 1141 | cacagacggc gcgggacttg caggagctgc gaggtcgggc ggatgagctg tgcaccatgg |
| 1201 | tggagcggtc agcagtgtct gtggcttcac tgaggagcga actggagggg ctgggcccac |
| 1261 | tgaaacccat tctggaggag ttcgggcggc aatttcagaa ctctcgaaga gggcctgacc |
| 1321 | tctccatgaa cctggatcgg tcccaccaag gcaactgtgc ccgctgtgcc agccaggggt |
| 1381 | cgcagttgtc tacggagtcc ctgcagcagc tgctggaccg agcactgacc tcactagtgg |
| 1441 | acgaggtgaa gcagaggggc ctgactcctg cctgtcccag ctgtcagagg ctacacaaga |
| 1501 | agattctgga gctggagcgc caggccttag ccaaacacgt cagggcagag gccctgagct |
| 1561 | ccacccttcg gctggcccaa gacgaggccc tgcgggccaa gaacctactg ctgacagaca |
| 1621 | agatgaagcc agaggagaag atggccactc tggaccatct acacttgaag atgtgctccc |
| 1681 | tccacgatca tctcagcaac ctgccacttg aggggtccac gggaacaatg gggggaggca |
| 1741 | gcagtgcagg aaccccccca aaacaggggg gctcagcccc tgaacaataa atggcctctc |
| 1801 | atgctagcat ga |

(SEQ ID NO:40)
MASVVVKTIWQSKEIHEAGDTPTGVESCSQLVPEAPRRVTSRAK
GIPKKKKAVSFHGVEPQMSHQPMHWCLNLKRSSACTNVSLLNLAAMEPTDSTGTDSTV
EDLSGQLTLAGPPASPTLPWDPDDADITEILSGVNSGLVRAKDSITSLKEKTNRVNQH
VQSLQSECSVLSENLERRRQEAEELEGYCIQLKENCWKVTRSVEDAEIKTNVLKQNSA
LLEEKLRYLQQQLQDETPRRQEAELQEPEEKQEPEEKQEPEEKQKPEAGLSWNSLGPA
ATSQGCPGPPGSPDKPSRPHGLVPAGWGMGPPAGEGPYVSEQELQKLFTGIEELRREV
SSLTARWHQEEGAVQEALRLLGGLGGRVDGFLGQWERAQREQAQTARDLQELRGRADE
LCTMVERSAVSVASLRSELEGLGPLKPILEEFGRQFQNSRRGPDLSMNLDRSHQGNCA
RCASQGSQLSTESLQQLLDRALTSLVDEVKQRGLTPACPSCQRLHKKILELERQALAK
HVRAEALSSTLRLAQDEALRAKNLLLTDKMKPEEKMATLDHLHLKMCSLHDHLSNLPL
EGSTGTMGGGSSAGTPPKQGGSAPEQ

| Mouse TSKS, NCBI accession number NM_011651- | |
|---|---|
| (SEQ ID NO:41) | |
| MASVVVKTIWQSKEIHEAGDPPAGVESRAQLVPEAPGGVTSPAK | |
| GITKKKKAVSFHGVEPRMSHEPMHWCLNLKRSSACTNVSLLNLAAVEPDSSGTDSTTE | |
| DSGPLALPGTPASPTTPWAPEDPDITELLSGVNSGLVRAKDSITSLKEKTTRVNQHVQ | |
| TLQSECSVLSENLERRRQEAEELEGYCSQLKGPRPDVLTQENCRKVTRSVEDAEIKTN | |
| VLKQNSALLEEKLRYLQQQLQDETPRRQEAELQELEQKLEAGLSRHGLSPATPIQGCS | |
| GPPGSPEEPPRQRGLSFSGWGMAVRTGEGPSLSEQELQKVSTGLEELRREVSSLAARW | |
| HQEEGAVQEPLRLLGGLGGRLDGFLGQWERAQREQAQSARGLQELRARADELCTMVER | |
| SAVSVASLRSELEALGPVKPILEELGRQLQNSRRGADHVLNLDRSAQGPCARCASQGQ | |
| QLSTESLQQLLERALTPLVDEVKQKGLAPASPSCQRLHKKILELERQALAKHVRAEAL | |
| SSTFGWPKTRPFGPRTYC | |
| (SEQ ID NO:42) | |
| 1 | acctgggagc aggccccccg caccatggca agcgtggtgg tgaagacaat ctggcaatcc |
| 61 | aaagagatcc acgaagcggg ggacccacct gcgggggtag aaagccgtgc ccagctggtc |
| 121 | cccgaggctc ccgggggggt gaccagccct gccaaaggga taacgaaaaa aaagaaggct |
| 181 | gtgtccttcc atggggtgga gccccggatg tcccacgagc cgatgcactg gtgcctgaac |
| 241 | ctcaagcggt cctctgcctg caccaacgtg tccttgctca acctggctgc cgtggagccc |
| 301 | gactcctcgg gcacagactc gaccacagag gacagtggtc cactggcact gccagggaca |
| 361 | cctgcttccc ctacaacacc ctgggctcca gaggaccctg acatcacaga actactgagt |
| 421 | ggggtcaaca gtggattggt acgtgccaaa gactccatca ccagcttgaa ggaaaagacc |
| 481 | acgcgggtta atcagcacgt tcagactctg cagagcgagt gctctgtgct gagtgagaat |
| 541 | ctggaaagaa gacggcagga ggcagaagag ttggaggggt actgcagtca gttgaagggc |
| 601 | ccccgccctg atgtcctgac ccaggagaac tgccgcaagg tgacccgttc agtggaagac |
| 661 | gctgaaatca aaaccaatgt cctgaagcag aactctgcct tgctggagga gaagctaaga |
| 721 | tacctccagc agcaactgca ggatgagacg ccccggagac aggaggccga gttgcaggag |
| 781 | ttggagcaaa aactggaggc tggcctctcc cgacatggtc tgagccctgc cactcccatt |
| 841 | cagggctgct cgggtcctcc tggcagcccc gaggaacccc cgcggcagcg aggcctgtcc |
| 901 | ttcagtggct ggggcatggc agtccgcaca ggggagggac cctcgctgag cgagcaggag |
| 961 | ttgcagaagg tctccaccgg cctggaggag ctgaggaggg aggtgtcctc gctggcagcc |
| 1021 | cggtggcatc aggaggaggg ggcagtgcag gagcccctga ggttgctggg aggccttggc |
| 1081 | ggccgtctgg atggcttcct gggccagtgg gagcgcgcgc agcgggaaca ggcacagtcc |
| 1141 | gcaaggggct tgcaggagct gcgagcacga gcagatgagt tgtgcactat ggtggagagg |
| 1201 | tcagcagtgt ctgtcgcctc actgaggagt gaactggagg cactgggccc agtaaaaccc |
| 1261 | attctggagg agctgggacg gcaacttcag aactcccgga ggggagctga ccatgtcttg |
| 1321 | aacttggatc ggtctgccca aggcccctgt gcgcgctgtg ccagccaggg gcagcagtta |
| 1381 | tccacggagt ccctgcagca gctgctggaa cgtgcgctga ccccgctggt ggatgaggtg |
| 1441 | aagcagaagg gtctggctcc tgccagcccc agttgccaga ggctacacaa gaagattctg |
| 1501 | gagctggagc gccaggcctt ggccaaacac gtcagggcag aggccctgag ctccaccttc |
| 1561 | ggttggccca agacgaggcc gttcgggcca agaacctact gctgacggac aagatgaagc |
| 1621 | cggaggagaa ggtggccact ttggactata tgcatttgaa gatgtgctcc ctccacgacc |
| 1681 | aactcagcca cctgccactt gaggggtcca cggggcaatg gggggaggaa gcaatggagg |
| 1741 | ggctccccct aaacgtggga gtccaggctc tgaacaataa atggcctctc atgctggcat |
| 1801 | gaaaaaaaaa aaaa |

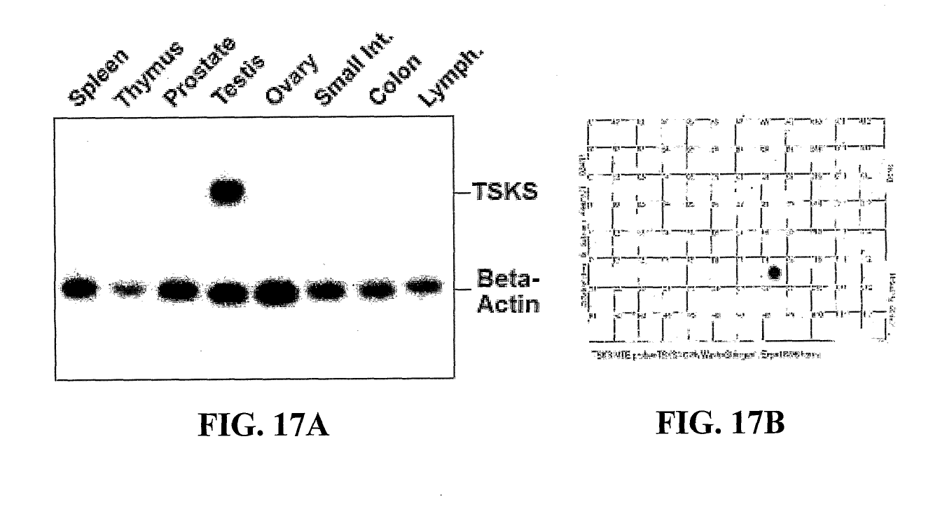

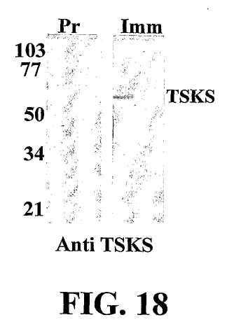

Northern (FIG. 17A; eight human tissues) and dot (FIG. 17B; 72 human tissues) blot analyses of TSKS expression were performed, finding testis specific and post-meiotic expression of human TSKS. TSKS transcripts were only detected in testis.

Western blot analysis of TSKS expression found TSKS protein in human testis but not in mature spermatozoa.

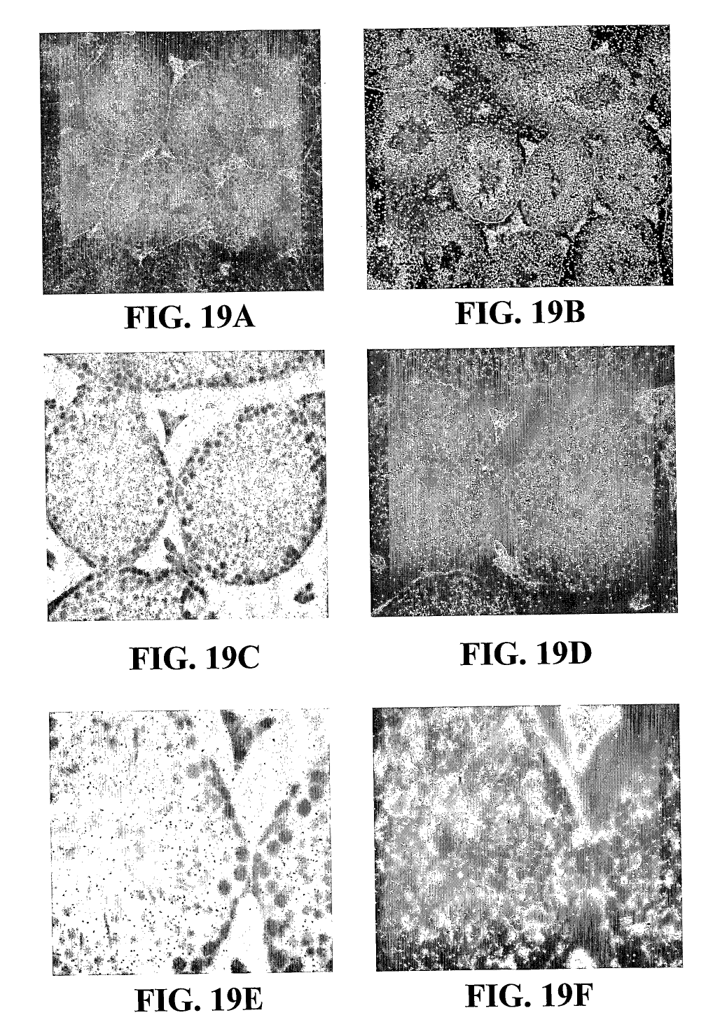

In situ hybridization of TSKS transcripts was carried out using radiolabeled mouse TSKS cRNA in adult mouse testis (see FIG. 19). TSKS transcripts were expressed mainly in the post-meiotic spermatids, and the labeling of the primary spermatocytes or spermatogonia is not much greater than the background.

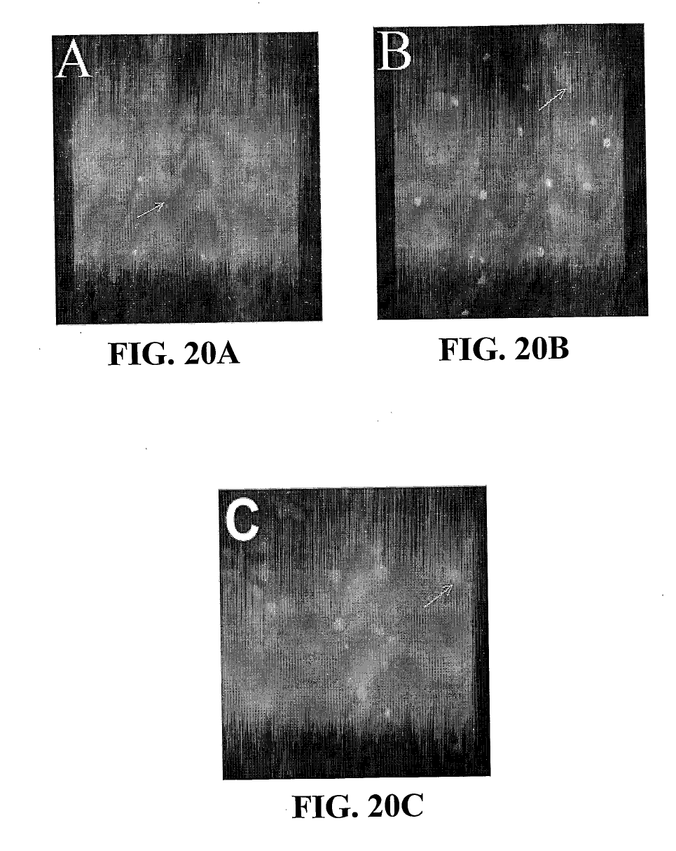

Immunofluorescent staining of TSKS in disassociated cells from human testis was performed. Human TSKS, red signal, was detected in round spermatids (FIG. 20A) as small immunofluorescent dots detached from the nuclei [blue, DAPI stained] at the acrosomal poles. In early and late elongating spermatids (FIG. 20B), TSKS immunofluorescence was similarly adjacent to the acrosomal pole and reached its peak intensity and size, suggesting association of TSKS with the Golgi apparatus. TSKS immunofluorescence was virtually absent in mature testicular sperm (20C).

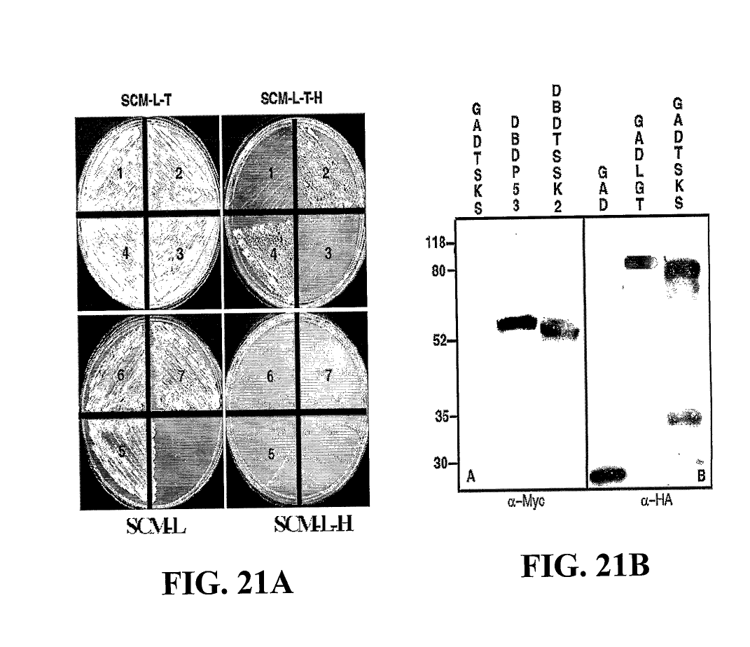

FIG. 21A depicts interaction of TSSK2 and TSKS in a yeast two hybrid system. Yeast host strain AH109 was transformed with either a pair of plasmids or a single plasmid as follows: pGAD, pGBK-TSSK2 (1); pGAD-TSKS, pGBK-TSSK2 (2); pGAD, pGBKp53 (3); pGAD-lgT, pGBK-p53 (4); pGAD (5); pGAD-TSKS (6); and pGAD-LgT (7). The transformants were streaked on complete drop-out media lacking both leucine and tryptophan (SCM-L-T), leucine (SCM-L), both leucine and histidine (SCM-L-H), or leucine, tryptophan and histidine (SCM-L-T-H) to test for histidine prototrophy. The p53 and the SV40 large T antigen controls (4) as well as TSSK2 and TSKS (2) interacted in the yeast two hybrid system.

FIG. 21B represents confirmation of hybrid protein co-expression by western blotting. ORFs of TSSK2 and TSKS were fused with GalDBD and GalAD respectively and co-transformed into AH109 host strain. The strains were tested for expression of DBD-TSSK2 (myc tagged), AD-TSKS (HA tagged). DBD-P53 (myc tagged) and GAD-LgT (HA tagged) were used as positive controls. Both TSSK2 and TSKS were co-expressed in AH109 validating this model for further studies of binding interaction.

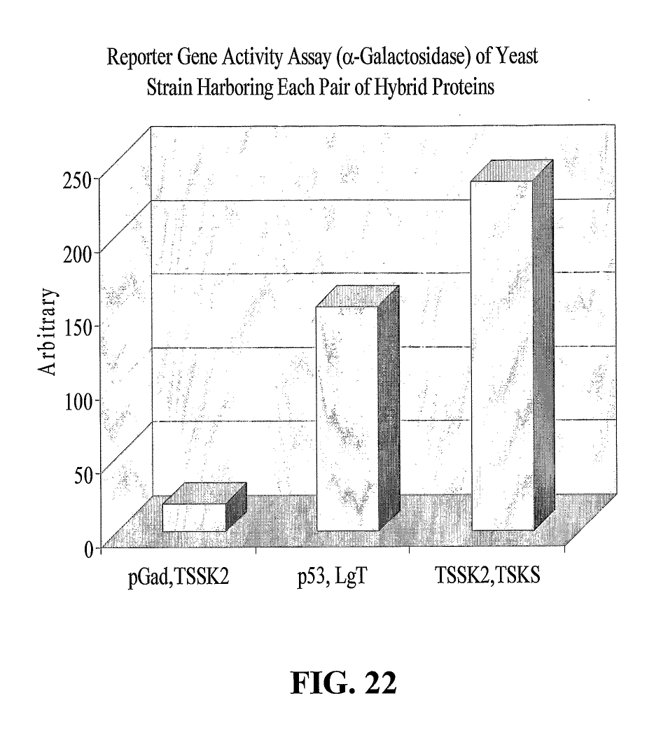

FIG. 22 graphically illustrates a reporter gene activity assay (α-galactosidase) of a yeast strain harboring each pair of hybrid proteins, as measured by the quantitation of the binding strength between TSSK2 and TSKS. Alpha-galactosidase activity was measured at OD 410 by incubating the culture supernatants of AH109 harboring each pair of plasmids as indicated using r-nitrophenyl-a-D-Galactopyranoside as a substrate. The activities were expressed as arbitrary units after calibration the optical density of the culture supernatants. TSKS binding strength with TSSK2 was 12 times stronger than the negative control while being 1.5 times stronger than the positive control interaction between p53 and LgT.

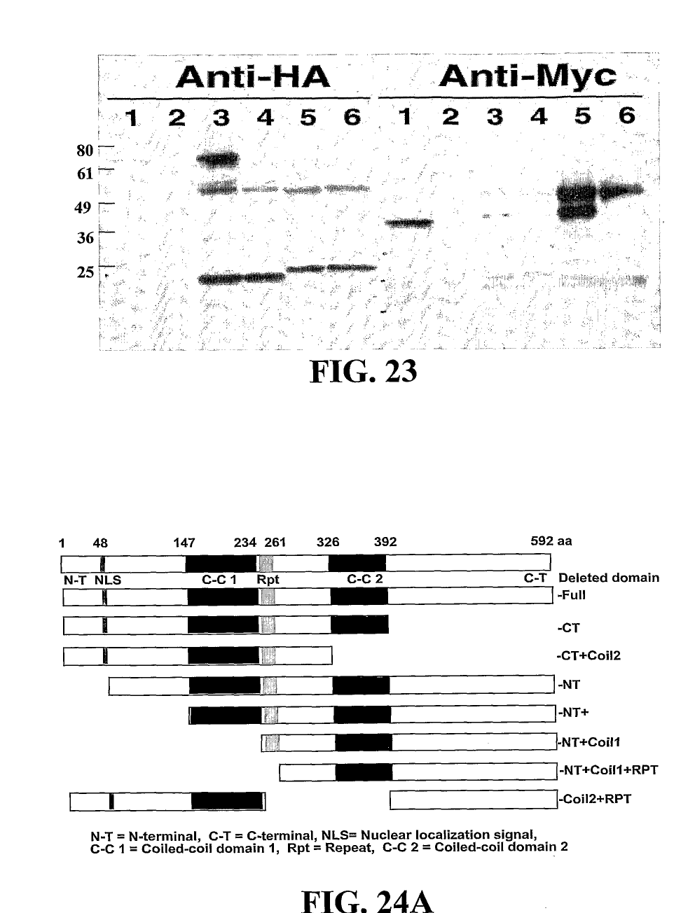

An electrophoretic analysis demonstrating co-immunoprecipitation of human TSSK2 with human TSKS in vitro is demonstrated in FIG. 23. TSSK (myc tagged) and TSKS (HA tagged) were co-translated in a Promega in-vitro translation kit. The mixture was immunoprecipitated with either agarose beads coupled to monoclonal antibody against myc (mice) or HA (rat). Immunoprecipitations were separated with SDS-PAGE and blotted with either anti-HA (left) or anti-myc (right): 1. In vitro translation mix; 2. In vitro translation control; 3. IP (anti-HA); 4. HA-IP control; 5. IP (anti-Myc); and 6. Myc-IP control. It can be seen that using either anti-myc or anti-HA, TSSK and TSKS co-immunoprecipitate, confirming their interactions.

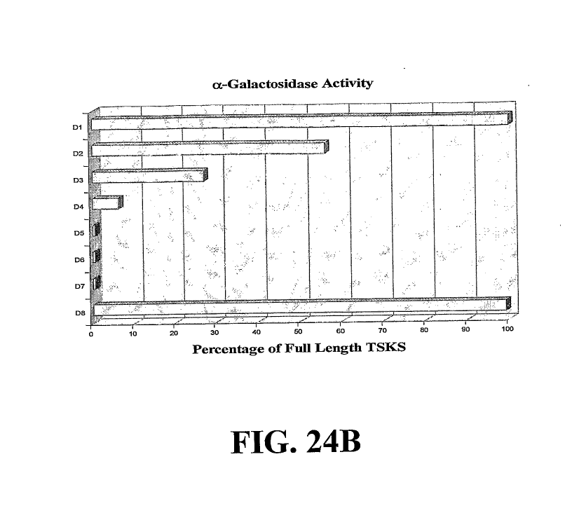

An analysis of the essential interacting domains of TSKS required for interaction with TSSK was performed. FIG. 24A represents a schematic drawing of TSKS ORF (top panel). Two putative coiled-coil domains [CC1, CC2] are filled. The six amino-acid repeats are hatched, and the amino terminus nuclear localization signal is indicated in gray. A total of 8 deletion mutants of TSKS were created and fused with the Gal4 activation domain in the pGAD two hybrid vector. Fusion proteins of each mutant and the Gal-AD were co-transformed with Gal-DBDTSSK2 into the yeast host strain AH109. FIG. 24B represents culture supernatants of each pair which were assayed for alpha-galactosidase activity and the activity measured for each mutant is expressed as percentage of full length TSKS. The corresponding deletion mutant is indicated on the ordinate. Deletion of the first 48 amino acids reduces TSKS binding to 5%, while deletion of the first 147 amino acids abolishes TSKS binding. Deletion of the carboxyl terminus and second coiled-coil region reduces interaction by 75%.

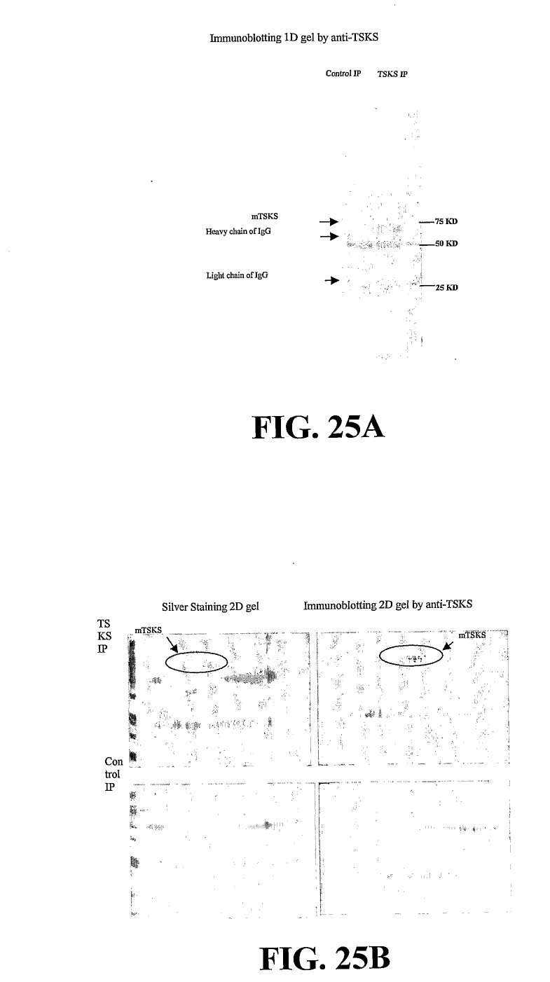

Studies of the phosphorylation of mouse TSKS in vitro were conducted. Immunoprecipitation of TSKS from mouse testis using rat antiserum against TSKS was performed. Precleared mouse testis extracts were immunoprecipitated with either rat normal serum (control IP), or rat anti-TSKS serum (TSKS IP), the immunoprecipitated complexes were separated on 1D (FIG. 25A) and 2D (FIG. 25B) gels, and subjected to silver staining and immunoblotting with anti-TSKS antibody. The results demonstrate that the rat anti-human TSKS serum was capable of immunoprecipitating mouse TSKS.

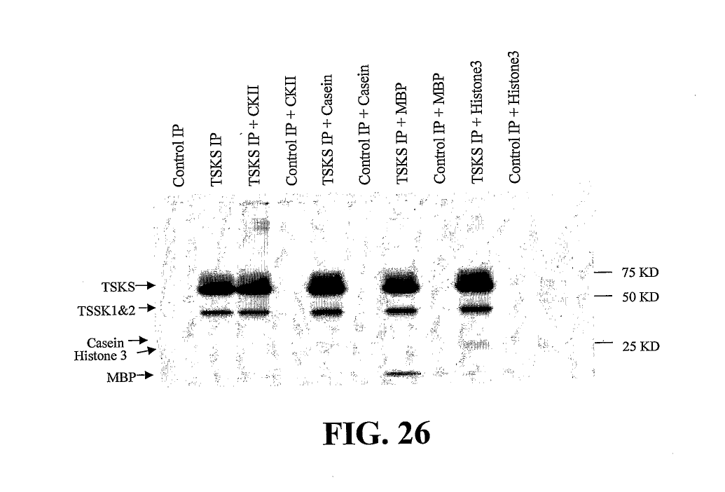

An electrophoretic analysis of in vitro phosphorylation of TSKS was performed (FIG. 26). Immune-complexes immunoprecipitated with either anti-TSKS (TSKS IP) or rat normal serum (control IP) and subsequently incubated with γP32 ATP in an in vitro kinase assay were subjected to autoradiography. A common kinase, casein kinase 2 (CKII) and several common kinase substrates including casein, maltose binding protein (MBP) and histone 3 were added to the reactions as the positive control. With anti-TSKS sera, TSKS was strongly phosphorylated as well as a 42 KD protein, while no proteins were phosphorylated with control normal rat sera. The 42 KD protein may be autophosphorylation of TSSK1 and/or TSSK2. Addition of CKII did not result in any additional phosphorylation indicating either phosphorylation of TSKS and TSSK is saturated or they are not substrates of CKII. Casein, MBP and histone3 were also phosphorylated by the kinases in the precipitated complex, whereas these proteins were not phosphorylated in the controls immunoprecipitated by normal sera. This kinase assay provides a high throughput screen for inhibitors of TSKS phosphorylation as well as demonstrating that casein, MBP and histone 3 may be used as alternative substrates for TSSK1 and TSSK2.

### Summary

TSKS, the substrate of TSSK 1 and 2, is a testis specific gene product, implying that tissue specific contraceptive targeting may be possible.

TSKS proteins are restricted to round and elongating spermatids, and their concentration is significantly reduced in mature sperm from the levels found in elongating spermatids. Their concentration appears to reach a peak in the elongating spermatids where they localize as discrete spots [likely Golgi apparatus] at the apical end of the sperm head adjacent to the acrosome. This maximal expression in elongating spermatids and subsequent diminution of expression suggests a role for TSKS during acrosome biogenesis.

Binding interactions between human TSSK2 and TSKS were confirmed by the yeast two hybrid system, and in-vitro co-immunoprecipitation.

Deletion of the first 48 amino acids reduces TSKS binding with TSSK2 to 5%, while deletion of the first 147 amino acids abolishes TSKS binding with TSSK2 entirely. Deletion of the carboxyl terminus and second coiled-coil region reduces interaction by 75%.

TSKS is post-meiotic in its expression pattern, which suggests a possible application in reversible contraceptive intervention by preserving spermatogonia and spermatocytes.

Mouse TSKS immunoprecipitated from testis was shown to be phosphorylated in an in-vitro kinase assay. This kinase assay provides a high throughput screen for inhibitors of TSKS phosphorylation.

The invention should not be construed to be limited solely to the assays and methods described herein, but should be construed to include other methods and assays as well. One of skill in the art will know that other assays and methods are available to perform the procedures described herein.

Headings are included herein for reference and to aid in locating certain sections. These headings are not intended to limit the scope of the concepts described therein under, and these concepts may have applicability in other sections throughout the entire specification.

The disclosures of each and every patent, patent application, and publication cited herein are hereby incorporated herein by reference in their entirety.

While this invention has been disclosed with reference to specific embodiments, it is apparent that other embodiments and variations of this invention may be devised by the previous description of the disclosed embodiments is provided to enable any person skilled in the art to make or use the present invention. Various modifications to these embodiments will be readily apparent to those skilled in the art, and the generic principles defined herein may be applied to other embodiments without departing from the spirit or scope of the invention. The appended claims are intended to be construed to include all such embodiments and equivalent variations. Accordingly, the present invention is not intended to be limited to the embodiments shown herein but is to be accorded the widest scope consistent with the principles and novel features disclosed herein.

What is claimed is:

1. A method of inhibiting fertilization, said method comprising contacting a sperm with a composition comprising at least one inhibitor selected from the group consisting of a tssk kinase inhibitor, a tsks inhibitor, and an inhibitor of tssk kinase and tsks interaction, thereby inhibiting fertilization.

2. The method of claim 1, wherein said tssk kinase comprises an amino acid sequence selected from the group consisting of SEQ ID NOs:4, 5, 6, and 20, and fragments and homologs thereof.

3. The method of claim 1, wherein said tsks comprises an amino acid sequence comprising SEQ ID NO:40, and fragments and homologs thereof

4. The method of claim 1, wherein said inhibitor of a tssk kinase inhibits synthesis, production, formation, accumulation, or function of said tssk kinase.

5. The method of claim 4, wherein said tssk kinase inhibitor inhibits phosphorylation of a tsks by said tssk kinase.

6. The method of claim 1, wherein said inhibitor of a tsks inhibits synthesis, production, formation, accumulation, or function of said tsks.

7. The method of claim 1, wherein said sperm is a human sperm.

8. A method of decreasing fertility in a subject, said method comprising administering to said subject a pharmaceutical composition comprising an effective amount of at least one inhibitor selected from the group consisting of a tssk kinase inhibitor, a tsks inhibitor, and an inhibitor of tssk kinase and tsks interaction, thereby inhibiting fertilization in said subject.

9. The method of claim 8, wherein said inhibitor is administered via a route selected from the group consisting of local, topical, oral, vaginal, intramuscular, nasal, and intravenous.

10. The method of claim 8, wherein said pharmaceutical composition is selected from the group consisting of a lotion, an aerosol, a cream, a gel, a liniment, an ointment, a paste, a solution, a powder, a tablet, and a suspension.

11. The method of claim 8, wherein said inhibitor is administered as a controlled-release formulation.

12. The method of claim 8, wherein said tssk kinase inhibitor inhibits interaction of said tssk kinase with a tsks.

13. The method of claim 12, wherein said tssk kinase inhibitor inhibits phosphorylation of said tsks.

14. The method of claim 8, wherein said subject is a male.

15. The method of claim 8, wherein said inhibitor of tsks inhibits a function selected from the group consisting of tsks synthesis, production, formation, accumulation, and function.

16. The method of claim 8, wherein said inhibitor of tssk kinase inhibits a tssk kinase having an amino acid sequence selected from the group consisting of SEQ ID NOs:4, 5, 6, and 20, and fragments and homologs thereof.

17. The method of claim 8, wherein said inhibitor of tsks inhibits a tsks having an amino acid sequence comprising SEQ ID NO:40, and fragments and homologs thereof.

18. The method of claim 8, wherein said inhibitor is an antibody.

19. The method of claim 18, wherein said antibody is a monoclonal antibody.

20. A method of decreasing fertility in a subject, said method comprising administering to said subject a pharmaceutical composition comprising an immunogenic amount of at least one protein or immunogenic fragment thereof of a tssk or tsks protein, thereby decreasing fertility in a subject.

21. The method of claim 20, wherein said protein has a sequence selected from the group consisting of SEQ ID NOs: 4, 5, 6, 20, and 40.

22. A recombinant gene construct for use in preparing a double knockout, said construct comprising a recombinant wild type TSSK1 and TSSK2 locus, wherein said TSSK1 and TSSK2 are deleted, further wherein remaining adjacent 5′ and 3′ nucleic acids are ligated into a knockout construct, further wherein said knockout construct is further submitted to homologous recombination and to neogene removal.

23. A transgenic cell comprising the construct of claim 22.

24. The transgenic cell of claim 23, wherein said cell is an embryonic stem cell.

25. A kit for inhibiting tssk function in a subject, said kit comprising an effective amount of at least one inhibitor of tssk function, said kit comprising an inhibitor of said tssk function, a standard, an applicator, and an instructional material for the use thereof.

26. A kit for inhibiting tsks function in a subject, said kit comprising an effective amount of at least one inhibitor of tsks function, said kit comprising an inhibitor of said tsks function, a standard, an applicator, and an instructional material for the use thereof.
